(19) United States
(12) Patent Application Publication
(10) Pub. No.: US 2026/0295220 A1
(43) Pub. Date: Oct. 1, 2026

(54) Catheter Insertion Device Having Continuous Flashback

(71) Applicant: TELEFLEX MEDICAL LLC, Morrisville, NC (US)

(72) Inventors: Benjamin Horst, Lititz, PA (US); Jeffrey Kuehn, Schuylkill Haven, PA (US); Luyando Chibwe, Shillington, PA (US)

(21) Appl. No.: 19/631,819

(22) Filed: Mar. 27, 2026

Related U.S. Application Data
(60) Provisional application No. 63/779,990, filed on Mar. 28, 2025.
(60) Provisional application No. 63/780,002, filed on Mar. 28, 2025.
(60) Provisional application No. 63/780,101, filed on Mar. 28, 2025.

(51) Int. Cl. A61M 25/06; A61M 25/09

(52) U.S. Cl. CPC A61M 25/0693; A61M 25/0618; A61M 25/065; A61M 25/09; A61M 25/09041; A61M 2205/586

(57) ABSTRACT

A catheter insertion device and a method of operating the device is disclosed. The device includes an inner body, a needle attached to the inner body, and an outer body. Moreover, the device includes a guidewire fixed to a proximal end of the outer body and a stiffening member fixed to the proximal end of the outer body configured to stabilize the guidewire when the outer body moves between the first position and the second position. Further, when the outer body moves between the first position and the second position, the outer body slides over the inner body and the guidewire and the stiffening member simultaneously move through an inner portion of the inner body. Additionally, when the outer body is at the first position, a tip of the guidewire is within the needle and at the second position, this tip is advanced into a vessel of a patient.

### CROSS-REFERENCE TO RELATED APPLICATIONS

This application claims benefit of priority to U.S. Provisional Application No. 63/779,990, filed Mar. 28, 2025, and U.S. Provisional Application No. 63/780,002, filed Mar. 28, 2025, and U.S. Provisional Application No. 63/780,101, filed Mar. 28, 2025, the entire contents of which are incorporated by reference herein.

### FIELD OF THE INVENTION

The present disclosure generally relates to a catheter insertion device operable to provide continuous pulsatile blood flashback during catheterization.

### BACKGROUND

Catheters are commonly used to remove fluids from blood vessels of a patient, or introduce fluids into blood vessels, for a variety of medical procedures. For instance, radial artery catheterization may be performed to provide access to the arterial system via the radial artery for monitoring blood pressure and withdrawing blood. In a typical catheterization procedure, for instance, in order to insert a catheter in a vessel, the vessel access is first performed by using a long hollow needle to pierce the vessel wall. A guidewire is then passed through the needle and into the vessel. The guidewire acts as a track for the catheter to pass over to reach a target location within the vessel. A catheter is finally passed over the guidewire to the target location in the vasculature of the patient. With the catheter in place, the needle and the guidewire are removed, leaving only the catheter within the vessel. Fluids may then be introduced into, or removed from, the vessel through the catheter by connecting a fluid source or aspiration device to the catheter hub.

Conventional catheterization devices include a means for the practitioner to visualize blood flash for indicating when the tip of the needle has entered the vasculature during vessel puncture. However, such conventional devices do not permit pulsatile blood flash in the device (i.e., the wire tube) during and after advancement of the guidewire. Additionally, such conventional devices do not offer the ability for blood flash to re-start after it is first observed, which makes it difficult for the practitioner to recover an insertion when the needle tip becomes misaligned with the vessel or passes through the vessel. Rather, the blood flash in conventional catheter insertion devices stops during and after the subsequent guidewire advancement step.

Other conventional catheterization devices offer the ability for users to visualize blood flash during vessel puncture through a transparent, or partially translucent, catheter chamber as the blood enters the small volume of space between the inside of the hollow catheter and the outside of the hollow needle. These devices show flash through the catheter when it initially enters into this small volume of space, but these devices do not offer the ability for flash to re-start after it is first observed and very quickly fills this small volume of space. For example, devices of this kind of funnel like shaped chambers which fill quickly on the distal end but then slows as the chamber fills toward the proximal end to the point where the flow rate of the blood flash is not helpful to the clinician. This aspect makes it difficult for the practitioner to recover an insertion in which the needle tip becomes misaligned with the vessel or passes through the vessel. Even for a catheter insertion device that includes pulsatile blood flash that remains visible in the device (e.g., the guidewire tube, handle or the like) after guidewire advancement, these devices do not provide adequate continuous flash visibility for clinical use. So, even if the annular cross-sectional area was increased from the use of a smaller guidewire, and the chamber was cylindrical, rather than funnel shaped, the guidewire tube would need to be increased in length to allow the user to adequately see the blood flash. However, increasing the length of the device would position the location where the clinician manipulates the guidewire much further back from the point where the clinician holds the device at the catheter hub and thus increase the guidewire reach length. Thus, this increase in the guidewire reach length would make the catheter insertion device very difficult to use. Furthermore, the mechanism for guidewire advancement would need to ensure it did not allow blood to escape from the guidewire tube (e.g., if the guidewire tube had an opening or slit or the like as is on conventional devices); otherwise, it would prevent the continuous flash visibility after guidewire advancement.

Therefore, a clear need exists for a novel catheter insertion device that allows for continuous visible blood flashback during insertion of a catheter into the vasculature of a patient. More particularly, there exists a need for a catheterization device that offers the ability to visualize flash through the catheter and flash in the device (i.e., the guidewire tube or the like) (which also includes the ability to re-confirm flash in guidewire tube or the like), and also offer the ability to visualize flash during and after guidewire advancement.

There is also a need for a catheterization device that offers the ability to visualize blood flash in the guidewire tube or the like (which also includes the ability to re-confirm flash in the guidewire tube or the like) and also start the guidewire inside the needle and near the needle tip, which both reduces guidewire reach length and reduces guidewire advancement dead length. These features thus improve the ease and speed of catheterization by allowing the guidewire to enter and secure the artery for catheterization and offer visual feedback to the user through blood flash. Additionally, a need exists for a catheter insertion device that allows for intuitive, easy, safe, and fast catheter placement into a patient's vasculature.

Moreover, known guidewire advancers may require a significant force to actuate the advancer to move the guidewire forward, which may cause the device to be dislodged or without a tactile feel to aid the clinician in accurately placing the guidewire in the vessel. Other guidewire advancers may rather allow the guidewire to freely slide, so if the clinician holds the device upright before the needle punctures the skin, the guide wire may fall forward from gravity which may increase the likelihood of damage to the patient's skin or the catheter insertion device. Thus, a need exists for a catheter insertion device that provides for stable and reliable catheter placement into a patient's vasculature by providing a sufficient amount of resistance to the guidewire during guidewire advancement to ensure the guidewire is not free to slide but also provides enough of a tactile feel to the clinician to prevent dislodging.

Additionally, in clinical practice, injuries from needles are dangerous because of blood and infective agents that contaminate the used needle. These injuries, which are occupational hazards of the medical profession, have resulted in inadvertent transmission of infective agents such as hepatitis C and human immunodeficiency virus. Sharps collection boxes, gloves and various safety practices are used to mitigate injuries arising from used needles, and to prevent infections from the needles. However, the presently available safety devices for protecting against injuries and infections resulting from needles are not completely fail-safe. Moreover, radial artery catheters often have integrated guidewires which many existing needle safeties do not work with. Furthermore, conventional safety clips that work with guidewires are often too large and thus cause the needle housing to be too big or bulky, which provides for poor device ergonomics and increases the likelihood of adverse behavior of the safety clip during catheter insertion. Therefore, the present disclosure addresses these unmet needs by providing a new safety clip for a needle that is compatible for use with a guidewire, provides for improved ergonomics, minimizes the risk of poor safety clip performance, and is easy to manufacture.

Therefore, a need exists for a novel catheter insertion device that allows for continuous visible pulsatile blood flashback during insertion of a catheter into the vasculature of a patient. More particularly, there exists a need for a catheter insertion apparatus that offers the ability to visualize the continuous pulsatile blood flashback in the guidewire tube both during and after guidewire advancement. Furthermore, a need exists for a catheter insertion device that has the ability to re-start blood flashback in the guidewire tube after initial flashback has stopped. Additionally, a need exists for a catheter insertion device that allows for intuitive, easy, safe, and fast catheter placement into a patient's vasculature.

### SUMMARY OF THE INVENTION

As used herein, including the appended claims, the singular forms of words such as “a,” “an,” and “the” include their corresponding plural references unless the context clearly dictates otherwise. All references cited herein are incorporated by reference to the same extent as if each individual publication, patent, and published patent application, as well as figures and drawings in said publications and patent documents, was specifically and individually indicated to be incorporated by reference.

The present disclosure solves the foregoing problems and relates to a catheter insertion device and method of use discussed herein. For example, in one aspect, a catheter insertion device may include an inner body defining a continuous pulsatile blood flashback chamber. The catheter insertion device may also include a needle hub having a proximal end attached to a distal end of the inner body. The catheter insertion device may furthermore include a hollow introducer needle extending from a distal end of the needle hub, the introducer needle having a tip configured to pierce a wall of a blood vessel. The catheter insertion device may in addition include a catheter configured to slidably fit over the introducer needle, the catheter extending from a catheter hub. The catheter insertion device may moreover include an outer body configured to slidably receive a portion of the inner body. The catheter insertion device may also include a guidewire coupled to a proximal end of the outer body. The catheter insertion device may furthermore include a stabilizer coupled to a proximal end of the inner body, the stabilizer having a passage configured to receive the guidewire. The catheter insertion device may in addition include where the outer body is further configured to move between a first position in which the guidewire is at an undeployed state and a second position in which the guidewire is at a deployed state. Moreover, a tip of the guidewire may be located within the needle when the outer body is at the first position; the tip of the guidewire may be advanced beyond the tip of the needle when the outer body is at the second position; and the passage of the stabilizer may be configured to provide frictional resistance to the guidewire when the outer body moves between the first position and the second position.

Implementations of the disclosure may also include one or more of the following features.

The passage of the stabilizer may be further configured to have an axis angularly offset by an angle with respect to a longitudinal axis of the catheter insertion device, where the angle is between 3 and 10 degrees.

The stabilizer may include a projection, the outer body may include an elongated channel, and the projection is configured to slide within the elongated channel to prevent or minimize rotation of the outer body relative to the inner body when the outer body moves between the first position and the second position.

The outer body further may include a stop member at a distal end of the elongated channel configured to abut the projection of the stabilizer to prevent the outer body from moving proximally relative to the inner body.

The stop member may include a resilient finger forming a terminus of the elongated channel.

The projection of the stabilizer may be configured to elastically deflect the finger upon application of a threshold force on the longitudinal axis of the outer body to allow the outer body to detach from and/or attach to the inner body.

A distal end of the outer body may include a guidewire actuator configured to move the guidewire between the undeployed state and the deployed state.

The catheter insertion device may include a safety clip configured to provide sharps protection to the tip of the introducer needle upon removal of the catheter from the introducer needle.

The safety clip may include a proximal portion having a first proximal wall directly connected to a second proximal wall at a clip bend, the first proximal wall may define a first proximal aperture and the second proximal wall defining a second proximal aperture, and the second proximal wall may be more proximal than the first proximal wall. The safety clip may have a first distal portion including a first distal wall, the first distal wall defining a first distal aperture for receiving the needle.

The safety clip may have a first clip arm may extend from the first distal portion to the first proximal wall and may have a medial aperture for receiving the needle. The safety clip may have a second distal portion having a second distal wall, the second distal wall defining a second distal aperture for receiving the needle. The safety clip may have a second clip arm extending from the second distal portion to the second proximal wall.

The first clip arm and the second clip arm may be in contact with the needle, and the first clip arm and the second clip arm may be configured to flex between a first configuration and a second configuration to move the safety clip relative to the needle. When the first clip arm and the second clip arm are in the first configuration, the safety clip may be able to slide along a length of the needle and the first distal wall and the second distal wall are spaced apart. When the first clip arm and the second clip arm are in the second configuration, the second proximal aperture may be configured to engage a swaged portion of the needle such that the first distal wall and the second distal wall overlap to cover a tip of the needle.

The first clip arm and the second clip arm may not intersect in either the first configuration or the second configuration.

The safety clip further may include a keeper configured to provide frictional resistance against an inner surface of the catheter hub to retain the safety clip inside the catheter hub.

The catheter hub further may include a bump configured to prevent the safety clip from being removed from the catheter hub in a first configuration and configured to allow the safety clip to be removed from the catheter hub in a second configuration.

The stabilizer may include a rudder, the outer body may include an elongated slot, and the rudder may be configured to slide within the slot to prevent or minimize rotation of the outer body relative to the inner body when the outer body moves between the first position and the second position.

The outer body further may include a stop member at a distal end of the elongated slot configured to abut the rudder of the stabilizer to prevent the outer body from moving proximally relative to the inner body.

The stop member may include a pair of protrusions extending into the slot, the pair of protrusions forming a passage to an open distal end of the slot; and where the rudder of the stabilizer is configured to pass through the passage of the slot upon application of a threshold force against the pair of protrusions to allow the outer body to detach from and/or attach to the inner body.

In another aspect of the disclosure, a method of using the catheter insertion device may be implemented. The method may include moving the outer body of the catheter insertion device to a first position. The method may also include inserting a needle attached to a proximal end of an inner body of the device into the vessel of the patient. The method may furthermore include moving the outer body from the first position, where a tip of the guidewire is within the needle, to a second position, where the tip of the guidewire is advanced into the vessel of the patient. In aspects, when the outer body moves between the first position and the second position, the outer body slides over the inner body and the guidewire moves through an inner portion of the inner body. In aspects, the guidewire may extend through a passage of a stabilizer and is fixed to a distal end of the inner body. The method may also the passage is configured to produce a frictional resistance on the guidewire when the outer body moves from the first position to the second position.

Aspects of the method of using the catheter insertion device may include one or more of the following features. For example, the aperture may be further configured to have an axis angularly offset by an angle with respect to a longitudinal axis of the catheter insertion device, where the angle is between 3 and 10 degrees.

The outer body further may include a stop member at a distal end of an elongated channel configured to abut the projection of the stabilizer to prevent the outer body from moving proximally relative to the inner body.

The stop member may include a finger forming a terminus of the elongated channel; and where the projection of the stabilizer is configured to elastically deflect the finger upon application of a threshold force on the longitudinal axis of the outer body to allow the outer body to detach from and/or attach to the inner body.

The outer body further may include a stop member at a distal end of the elongated slot configured to abut the rudder of the stabilizer to prevent the outer body from moving proximally relative to the inner body. The method where the stop member includes a pair of protrusions extending into the slot, the pair of protrusions forming a passage to an open distal end of the slot; and where the rudder of the stabilizer is configured to pass through the passage of the slot upon application of a threshold force against the pair of protrusions to allow the outer body to detach from and/or attach to the inner body.

The catheter insertion device further may include a safety clip configured to provide sharps protection to the tip of the introducer needle upon removal of the catheter from the introducer needle, where when the safety clip reaches a bevel of a tip of the needle, the safety clip activates and allows the first clip arm and the second clip arm to move from the first configuration to the second configuration.

While the catheter insertion device has been described in terms of what may be considered to be specific aspects, the present disclosure is not limited to the disclosed aspects. Additional modifications and improvements to the catheter insertion apparatus may be apparent to those skilled in the art. Moreover, the many features and advantages of the disclosure are apparent from the detailed specification, and thus, it is intended by the appended claims to cover all such features and advantages of the disclosure which fall within the spirit and scope of the disclosure.

### BRIEF DESCRIPTION OF THE DRAWINGS

In order that the disclosure may be readily understood, aspects of the catheter insertion device are illustrated by way of examples in the accompanying drawings, in which like parts are referred to with like reference numerals throughout.

FIG. 1 discloses a perspective view of a catheter insertion device according to an embodiment of the present invention in a first position.

FIG. 2 discloses a perspective view of the catheter insertion device according to an embodiment of the present invention in a second position.

FIG. 3 discloses a perspective view of the needle of the catheter insertion device being inserted into a vessel of a patient.

FIG. 4 discloses a side plan view of the catheter hub and the needle hub.

FIG. 5 discloses a perspective view of the stabilizer.

FIG. 6 discloses a side view of the stabilizer.

FIG. 7 discloses a front view of the stabilizer.

FIG. 8 discloses a cross sectional side view of the stabilizer along the line A-A.

FIG. 9 discloses a back view of the stabilizer.

FIG. 10 discloses a front plan view of the outer body.

FIG. 11 discloses a side plan view of the catheter insertion device in the first position.

FIG. 12 discloses an enlarged side plan view of the area indicated in FIG. 10 showing the profile of the guidewire installed in the catheter insertion device.

FIG. 13 discloses an enlarged side plan view of the area indicated in FIG. 10 showing the profile of the guidewire installed in the catheter insertion device.

FIG. 14 discloses an enlarged side plan view of the area indicated in FIG. 10 showing the profile of the guidewire installed in the catheter insertion device.

FIG. 15 discloses an enlarged side plan view of the area indicated in FIG. 10 showing the profile of the drag section of the guidewire and an override mechanism of the catheter insertion device.

FIG. 16 discloses a perspective view of the drag section of the guidewire installed in the catheter insertion device.

FIG. 17 discloses a perspective view of the override mechanism of the catheter insertion device.

FIG. 18 discloses an enlarged side plan view of an override mechanism including a stabilizer according to another aspect of the disclosure.

FIG. 19 discloses a perspective view of the stabilizer shown in FIG. 18.

FIG. 20 discloses a front view of the stabilizer shown in FIG. 18.

FIG. 21 discloses an enlarged side plan view of the area indicated in FIG. 11 showing the profile of the guidewire installed in the catheter insertion device.

FIG. 22 discloses a side view of the safety clip within the catheter hub of the catheter insertion device.

FIG. 23 shows a perspective view of the safety clip in the first configuration.

FIG. 24 shows a perspective view of the safety clip in the second configuration.

FIG. 25 shows a top plan view of the safety clip in the first configuration.

FIG. 26 shows a side plan view of the safety clip with a keeper in the first configuration.

FIG. 27 shows a side plan view of the safety clip with a keeper in between the first configuration and the second configuration.

FIG. 28 shows a side plan view of the safety clip with a keeper in the second configuration.

FIG. 29 shows a perspective view of the safety clip seen in FIG. 1 in an intermediate configuration.

FIG. 30 shows a side plan view of the safety clip in the first, open configuration.

FIG. 31 shows a side plan view of the safety clip in the second, closed configuration.

FIG. 32 shows a perspective view of the safety clip in the first, open configuration.

FIG. 33 shows a perspective view of the safety clip in the second, closed configuration.

FIGS. 34A-E show a side plan view of the catheter insertion device during use and retraction of the device.

FIG. 35 discloses a perspective view of a catheter insertion device according to an embodiment of the present invention in a first position.

FIG. 36 discloses a perspective view of the catheter insertion device according to an embodiment of the present invention in a second position.

FIG. 37 discloses a perspective view of the stabilizer.

FIG. 38 discloses a side view of the stabilizer.

FIG. 39 discloses a cross sectional top view of the stabilizer taken along the line B-B.

FIG. 40 discloses a front view of the stabilizer.

FIG. 41 discloses a back view of the stabilizer.

FIG. 42 discloses a front plan view of the outer body.

FIG. 43 discloses a bottom plan view of the catheter insertion device including the override mechanism.

FIGS. 44A and 44B discloses a top plan view of the outer body including the stabilizer and guidewire.

### DETAILED DESCRIPTION

As will be discussed in detail herein, the present disclosure describes an over-the-needle catheter insertion device operable to provide continuous pulsatile blood flashback visibility during catheterization. In particular, the catheter insertion device is operable to provide continuous pulsatile blood flashback in a guidewire passageway both during and after advancing the guidewire. The catheter insertion device is also operable to re-start pulsatile blood flashback within the guidewire passageway after initial pulsatile blood flashback within the guidewire passageway has stopped, i.e., due to misalignment of the needle tip within the vessel or due to passing the needle tip through the vessel wall. The ability to re-start pulsatile blood flashback in the guidewire passageway allows the practitioner to re-confirm proper placement of the needle tip within the patient's vasculature. The catheter insertion apparatus is also operable to provide the continuous blood flash visibility while starting the wire guide tip inside and near the needle tip, without causing excessive blood exposure, and without impacting other critical aspects of an insertion procedure.

The catheter insertion device of the present disclosure therefore improves insertion success rates and increases the confidence of practitioners in knowing that the needle tip is located inside the vessel throughout the entire insertion procedure without causing excessive blood exposure. The present catheter insertion apparatus may be utilized for catheterization of various vessels, including the radial artery or other arterial vessels, as well as the venous vasculature. Further, the present catheter insertion apparatus may be utilized to insert an introducer catheter used for facilitating the insertion of other catheters, such as a PICC (Peripherally Inserted Central Venous Catheter) or CVC (Central Venous Catheter), a peripheral IV catheter, or other vascular access devices.

In referencing the Figures and throughout the disclosure, like reference numerals are used to identify like components, while like reference numerals containing letter extensions are used to identify components that are different from other aspects of the disclosure. For brevity, the description of various components may not be repeated and will be understood to apply to all components unless the differences are otherwise described.

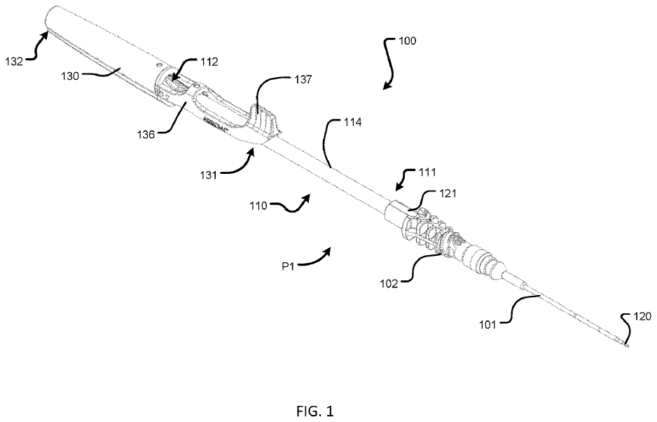

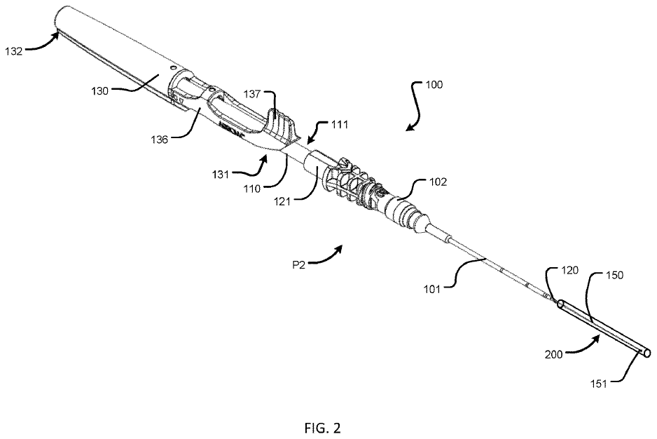

Referring to FIGS. 1 and 2, one aspect of the invention relates to a catheter insertion device 100 capable of continuous pulsatile blood flash which may include an inner body 110, which is designed to act as a pulsatile blood flashback chamber 114 to store blood of a patient during continuous flash. The catheter insertion device 100 capable of continuous pulsatile blood flash may also include a hollow introducer needle 120 extending from a distal end of the needle hub 121, the needle having a tip configured to pierce a wall of a blood vessel, and a guidewire 150. The catheter insertion device 100 capable of continuous pulsatile blood flash may furthermore include an outer body 130 configured to move between a first position P1 and a second position P2.

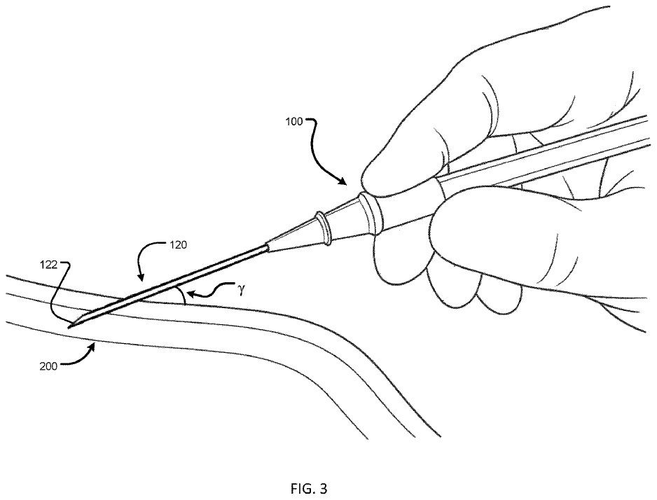

In this way, the catheter insertion device 100 may be configured to provide continuous pulsatile blood flashback within the flashback chamber 114 during catheterization, including before moving the guidewire 150, while moving the guidewire 150, and after moving the guidewire 150. In some aspects, the flashback chamber 114 is transparent so that the pulsatile blood flashback is visible to the user. In some aspects, the flashback chamber may include indicia, such as spaced apart alphanumeric markings or other type of markings, to indicate a specific distance of guidewire advancement. Further, FIG. 3 illustrates an insertion location for the catheterization procedure, where a practitioner inserts the tip 122 of the needle 120 at approximately a 30° insertion angle (γ) with respect to the surface of the skin to access a vessel 200 of the patient.

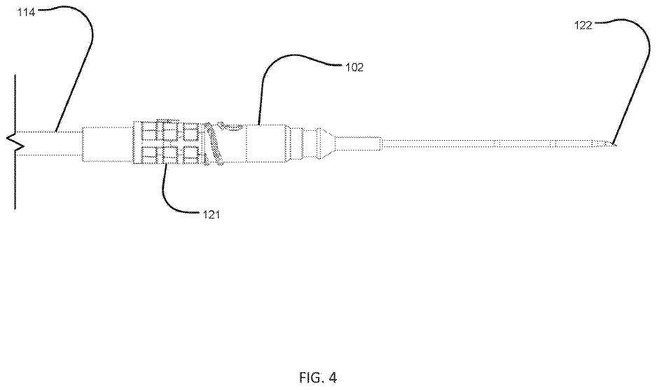

Additionally, the catheter insertion device 100 may include a catheter 101, such as an over-the-needle-catheter, including a catheter hub 102 removably coupled to a needle hub 121 of the catheter insertion device 100 and the needle hub 121 may be fixed to the distal end of the inner body 111 as shown in FIG. 4. For example, the catheter hub may be friction fit onto the needle hub, while the needle hub may be adhered onto the distal end of the inner body, such as by adhesive or other type of bonding. Further, the catheter may slidably fit over the introducer needle with the catheter extending from the catheter hub.

FIG. 1 shows the catheter insertion device 100 of the invention when it is in a first position P1. In this first position P1, the outer body 130 is at a distance extending away from the tip 122 of the needle 120 and the guidewire tip 151 is in an undeployed state located within the body of the needle 120. Additionally, in this first position P1 the distal end of the outer body 130 overlaps the proximal end of the inner body 112.

FIG. 2 shows the catheter insertion device 100 of the invention when it is in a second position P2. In this second position P2, the distal end of the outer body 130 is located close to the distal end of the inner body 111. Furthermore, while the outer body 130 is in the second position P2, the guidewire 150 is in a deployed state which extends past the tip 122 of the needle 120 and into the vessel 200 of a patient. In this way, the flashback chamber 114 may further define a portion of the guidewire passageway in which the guidewire 150 moves therein from the undeployed, retracted position to the deployed, extended position.

In another aspect of the invention, the outer body 130 of the catheter insertion device 100 may be configured to slidably receive a portion of the inner body 110 between the first position P1 and the second position P2. Further, the catheter insertion device 100 capable of continuous pulsatile blood flash may include a stabilizer 140 having an aperture 141. Additionally, a stabilizer 140 may be attached to a proximal end of the inner body 112 and thus the proximal end of the flashback chamber 114. The catheter insertion device 100 may moreover include a guidewire 150 attached to a proximal end of the outer body 132, which extends through the center of the inner body 110, the aperture 141 of the stabilizer 140, and the outer body 130 and into the needle 120.

Further, when the outer body 130 of the catheter insertion device 100 is at the first position P1, a guidewire tip 151 may be located within the needle 120 and when the outer body 130 is at the second position P2, the tip 151 the guidewire 150 may be advanced beyond the tip 122 of the needle.

Additionally, when the outer body 130 of the catheter insertion device 100 moves between the first position P1 and the second position P2, the outer body 130 may slidably receive a portion of the inner body 110.

Furthermore, the needle hub may be bonded to the inner body at its distal end, creating a fluid passageway from the tip 122 of the needle, through the needle 120, through the needle hub 121, and into the blood flashback chamber 114 of the inner body 110.

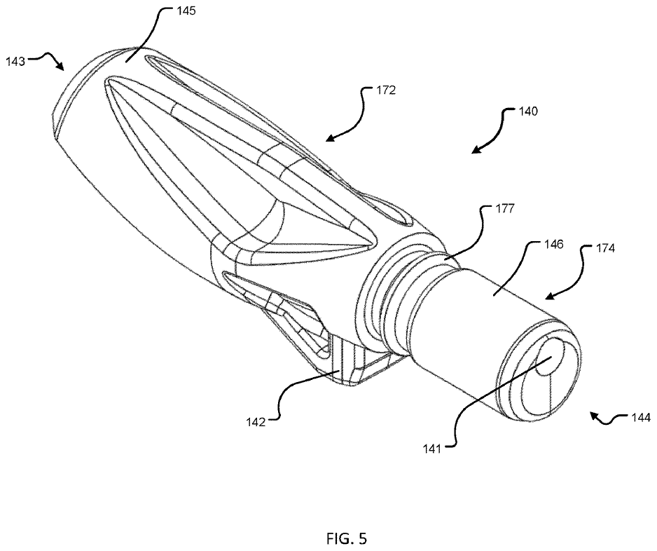

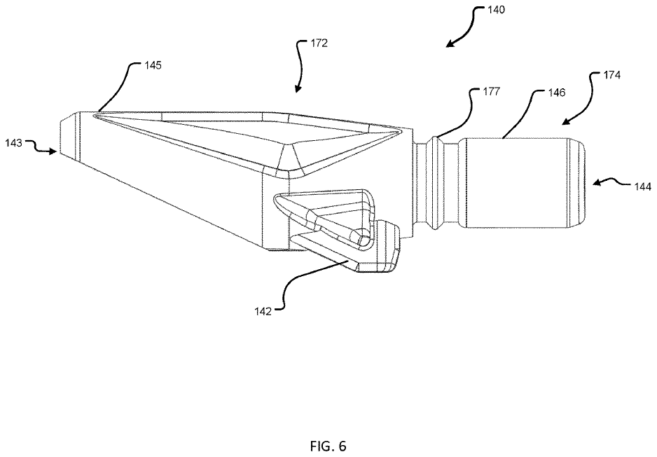

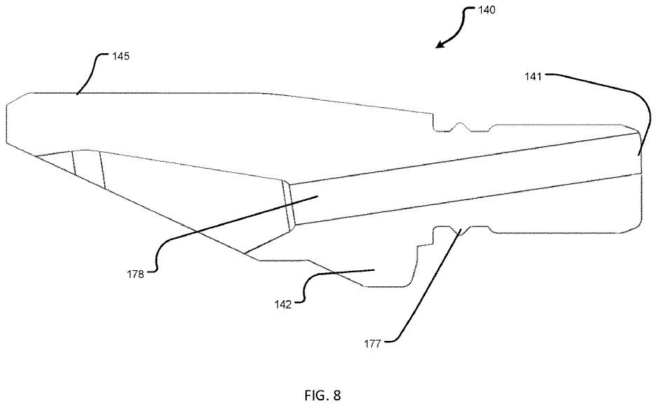

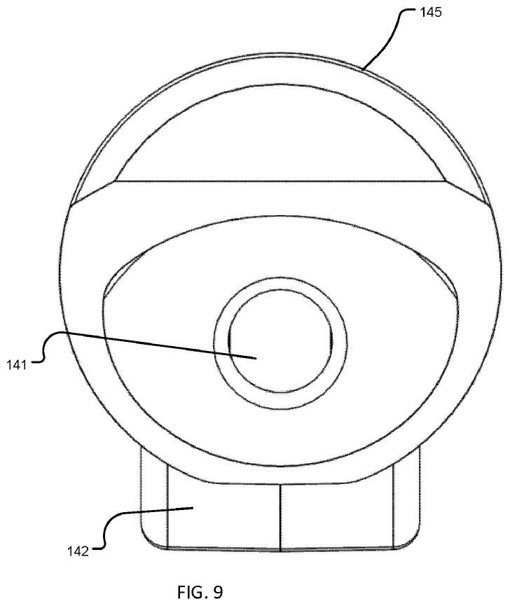

Referring to FIGS. 5-9, the stabilizer 140 of the catheter insertion device 100 may comprise a projection or rudder 142, the outer body 130 may comprise an elongated channel 133, and the projection 142 may be configured to slide along the elongated channel 133 to prevent or minimize the rotation of the outer body relative to the inner body when the outer body 130 moves between the first position P1 and the second position P2. The outer body 130 of the catheter insertion device 100 may further comprise a stop member 135 at a distal end of the channel 133, located at a distal end 131 of the outer body 130. The stop member 135 may be configured to abut against the projection 142 of the stabilizer 140 to prevent the outer body 130 from moving in a proximal direction relative to the inner body 130.

Moreover, the stop member 135 may be configured to provide a hard stop against the stabilizer 140 when the guidewire 150 is fully retracted. For instance, the stop member 135 may be configured to abut the projection 142 during retraction of the outer body 130.

In this respect, the stabilizer 140 may include a base 172, and a distal portion of the stabilizer 140 includes a nose 174. The projection 142 may extend from the base 172. An annular protrusion 177 is disposed between the base 172 and the nose 174. The nose 174 and the annular protrusion 177 are configured to be received within the proximal end of the flashback chamber 114 of the inner body 110. The annular protrusion 177 may be configured to provide an interference fit with an inner surface of the flashback chamber 114 for securing the stabilizer 140 thereto. The aperture 141 of the stabilizer may further comprise an angled passage 178 extending from the proximal portion to the distal portion such that the angled passage 178 extends through the nose 174 and the base 172. A proximal portion of the angled passage 178 may have a funnel shaped opening.

The outer body 130 includes a guidewire actuator 137 that is configured to move the guidewire 150 between the first and second positions P1, P2, as will be discussed in further detail below. The angled passage 178 of the stabilizer 140 is configured to receive and slide over the guidewire 150 as the guidewire actuator 137 is correspondingly moved. As the guidewire 150 passes through the angled passage 178 of the stabilizer 140, the guidewire 150 correspondingly bends which creates a spring force between the guidewire 150 and the angled passage 178 of the stabilizer 140. This configuration, in turn, provides a sufficient amount of frictional resistance between the guidewire 150 and the angled passage 178 of the stabilizer 140 as the guidewire 150 passes through the angled passage 178 of the stabilizer 140. Bending the guidewire 150 through the angled passage 178 of the stabilizer 140 therefore creates a drag force on the guidewire actuator 137 that prevents the guidewire actuator 137 from freely sliding back and forth over the flashback chamber 114 of the inner body 110. This drag force similarly prevents the outer body 130 from freely sliding back and forth over the flashback chamber 114 of the inner body 110. Stated another way, the angled passage 178 within the stabilizer 140 causes the guidewire to bend which creates a spring force between the guidewire and the stabilizer that provides an appropriate amount of drag (i.e., without excessive drag) to the guidewire actuator 137 in order to maintain desired control over guidewire advancement and retraction during a catheterization procedure. The frictional resistance between the guidewire 150 and the angled passage 178 of the stabilizer 140 therefore prevents the outer body 130 (including the guidewire actuator 137) from unintentionally sliding back and forth over the inner body 110 (including the flashback chamber 114) due to gravity or sudden movements of the catheter insertion device 100 by a user.

The stabilizer 140 may also comprise a proximal end 143 and a distal end 144. The stabilizer 140 may also comprise at least one surface configured to engage with at least one internal surface of the outer body 130 to prevent the inner body 110 from bowing or deforming when the outer body 130 reaches a finger 182 in the second position P2 as further described herein.

This aspect of the invention allows for ease of manufacturing and replacement of parts. By removing the outer body 130 from the inner body 110, the guidewire 150, and outer body 130 parts may be easily replaced if broken or damaged. Further, the projection 142 configured to slide along the channel 133 allows the catheter insertion device 100 to remain stable throughout the movement from the first position P1 to the second position P2. That is, the projection 142 prevents the outer body 130 from rotating around the inner body 110 while moving between the first position P1 and the second position P2.

Additionally, the outer body 130 of the catheter insertion device 100 may also comprise a guidewire actuator 137 (or advancer) on the distal end of the outer body 131 configured to move the guidewire 151 between the undeployed state as in the first position P1 and the deployed state as in the second position P2. As such, the guidewire actuator 137 may be configured to advance and retract the guidewire 150 relative to the needle 120. A distal end of the guidewire actuator 137 may include a flag (or handle) configured to allow a user to move the outer body between the first position and the second position. Further, the guidewire actuator 137 may be an over-molded portion 136 at a distal end of the outer body 130. In other implementations, the guidewire actuator 137 may also be snap fit, press fit, welded, glued, or otherwise attached to the outer body 130.

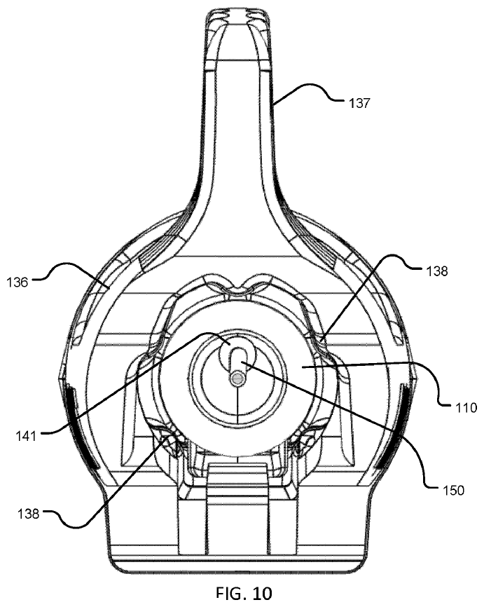

Here, the guidewire actuator 137 may include at least one projection extending inwardly from the outer body towards the inner body. Preferably the guidewire actuator 137 may include a plurality of projections 138, such as three or more projections or the five projections 138 seen in FIG. 10. This arrangement minimizes the contact points of the outer body on the inner body to create a smooth operation of movement when the outer body moves between the first position and the second position and provides minimal friction due to the limited number of contact points created between each projection 138 and the outer surface of the flashback chamber 114.

Further, the arrangement between the stabilizer 140, the angled passage 178, and the aperture 141, provides drag on the guidewire 150 as the outer body moves between the first position P1 and the second position P2. This configuration also helps to create a tactile feel to the user. As such, the location of the guidewire actuator 137 is configured to create a stable push point allowing the user to easily move the outer body from the first position P1 and the second position P2 without buckling the guidewire 150. The guidewire actuator 137 further provides for an advancement grip and may be located at the distal end of the outer body or as close to the front of the device as possible to provide for shorter reach and enable the user to use the device with one hand.

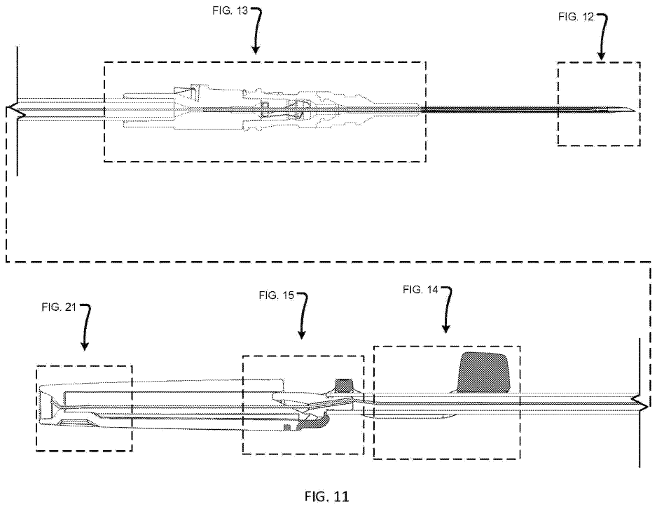

Referring to FIGS. 11-16, 18, and 21, in embodiments of the invention, the guidewire 150 may have a tip section 151, a first middle section 152, a second middle section 153, a drag section 154, and a proximal section 155. The guidewire 150 may extend from 0 to 4 centimeters from the tip 122 of the needle 120 during advancement. (e.g., when the catheter insertion device is at the second position P2).

Moreover, the guidewire 150 may be fixed at the proximal end of the outer body 132. The guidewire 150 may also be made of medical grade stainless steel, nitinol, or other materials known to those skilled in the art. The guidewire 150 of the present disclosure may be configured to be used with a 20-gauge catheter as described herein. However, in other aspects, the guidewire 150 may be configured to be used with various catheter gauges, such as 14-gauge to 26-gauge catheters, with respective increases or decreases in the diameter of the various sections of the guidewire 150 as would be understood to those skilled in the art.

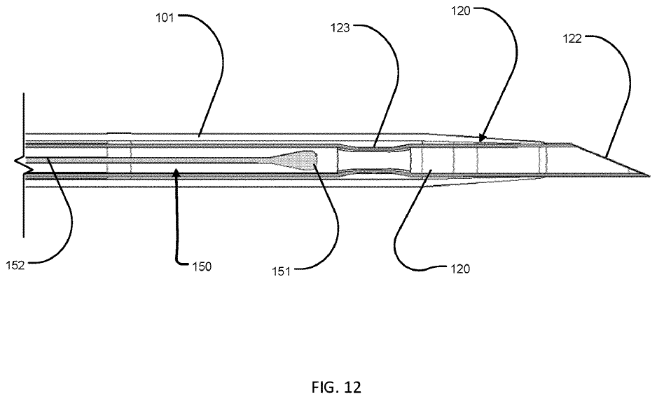

FIG. 12 illustrates the tip section 151 of the guidewire 150. The tip 151 may be tear drop shaped, ball shaped, rounded shape, or other shape known to those skilled in the art with a diameter between 0.013 to 0.018-inches, such as a 0.0165-inch diameter. The tip section 151 may be welded into shape on the guidewire 150, ground, or otherwise rounded through mechanical machining methods known to those skilled in the art. After the tip section 151 of the guidewire, the guidewire 150 decreases in diameter to a first middle section 152 between 0.003 to 0.007 inches, such as 0.0045 inches. Here, at the first middle section 152, the guidewire 150 may be configured to be floppy to allow for ease of advancement into the vessel 200 and reduce trauma to the vessel 200.

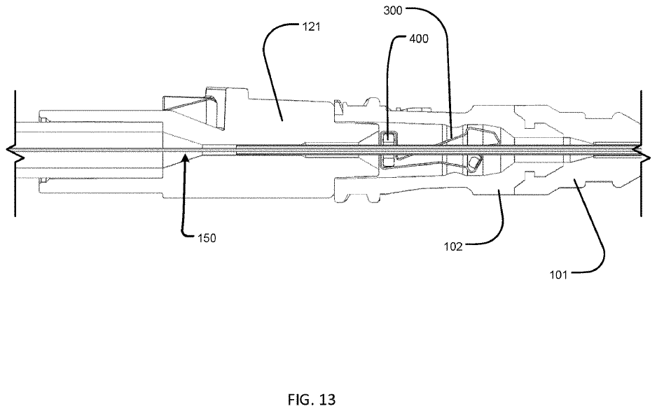

FIG. 13 illustrates a portion of the guidewire 150, which shows a second middle section 153 of the guidewire 150. Here, along the second middle section 153 of the guidewire 150, the first middle section 152 has tapered to the second middle section 153 from 0.0045 inches diameter to 0.0100 inches in diameter, for example. In other aspects of the invention, the diameter of the guidewire 150 in the second middle section 153 may be no greater than 0.007 to 0.015 inches, where the inner diameter of the needle 120 between 0.019 to 0.025 inches in diameter. This configuration ensures the annular volume, created by the inside of the needle 120 and the outside of the guidewire second middle section 153, is not too small that is prevents continuous pulsatile flash to flow from the tip 122 of the needle (when inserted into the vessel 200) to within the flashback chamber 114 within the catheter insertion device 100.

However, this configuration is non-limiting and one of ordinary skill in the art would understand the needle 120 and/or the guidewire 150 may be configured in various ways to ensure there is sufficient annular volume between the needle 120 and the guidewire 150 to still allow for continuous pulsatile flash. For example, in other aspects of the disclosure, the needle 120 may have grooves or channels cut into the inner diameter to create space for the blood to flow and allow for continuous pulsatile flash from the tip 122 of the needle to within the flashback chamber 114. In other aspects of the disclosure, the guidewire 150 may have grooves or channels cut into the outer diameter to create space for the blood to flow and allow for continuous pulsatile flash from the tip 122 of the needle to within the flashback chamber 114.

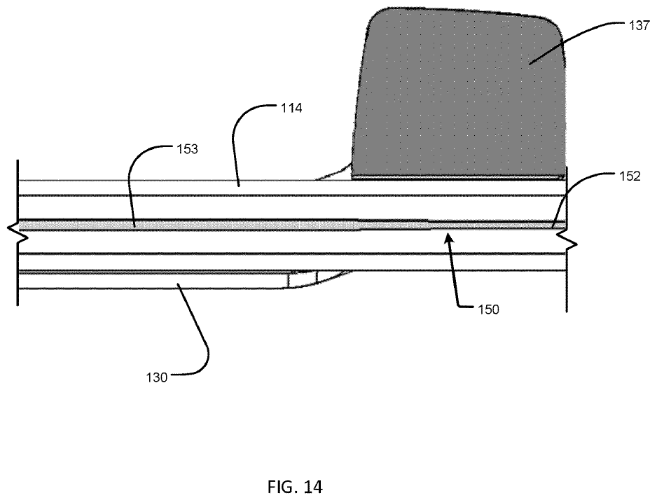

FIG. 14 illustrates a portion of the guidewire 150 as it transitions from the second middle section 153 to the drag section 154 of the guidewire 150. In this case, the guidewire 150 tapers along the second middle section 153 from its beginning 0.0100-inch diameter to the drag section 154 of a thickness of 0.0165-inch diameter.

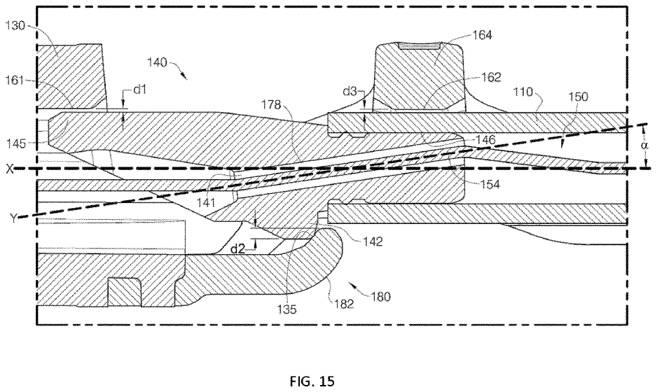

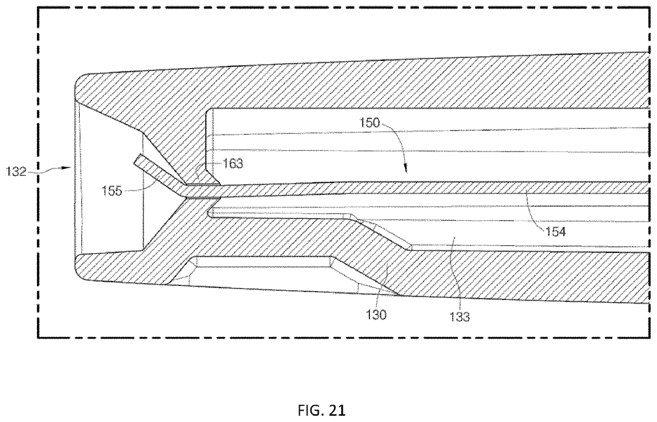

FIG. 15 illustrates a portion of the drag section 154 of the guidewire 150. In this section, the diameter may be uniformly 0.0320 inches to maintain a partial seal between the outer body 130, inner body 110, stabilizer 140, and guidewire 150. Thus, the drag section 154 extends from the portion seen in FIG. 14 to the proximal section 155 as seen in FIG. 21. The portion of the guidewire 150 may have a diameter of 0.0165 inches. This section of the guidewire is configured to slide through the angled passage 178 of the stabilizer 140. This proximal section of the guidewire is relatively stiffer than the other sections of the guidewire that are more distal to it. The stiffness of the proximal section of the guidewire allows it to provide a sufficient amount of spring force against the stabilizer when the proximal section of the guidewire is disposed within the angled passage 178. A distal section of the guidewire includes a gradual taper for increased flexibility. A distal end of the guidewire includes an atraumatic tip for preventing or minimizing damage to a vessel.

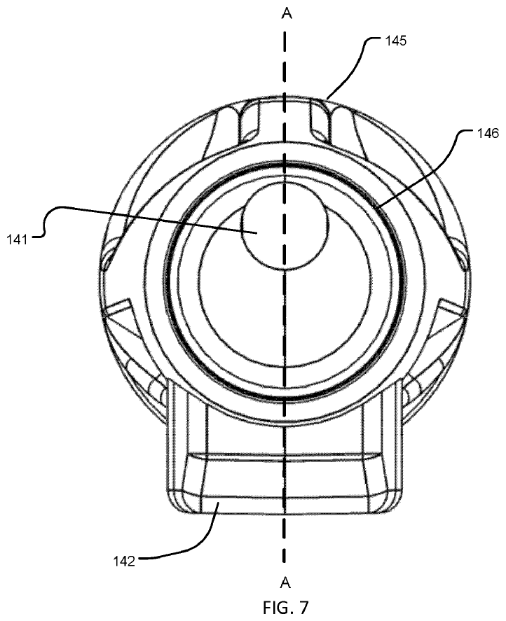

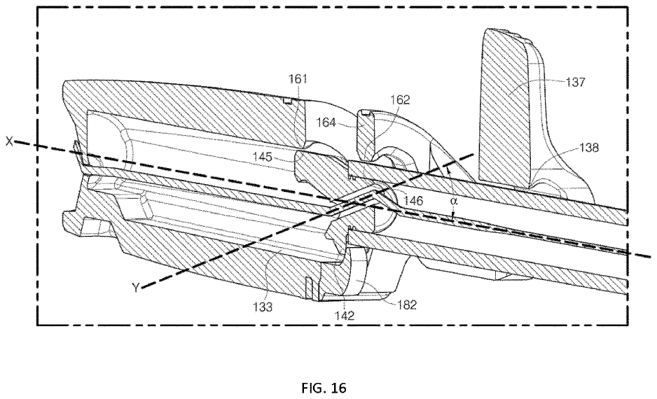

Further, as seen in FIGS. 15 and 16, the stabilizer 140 preferably has an aperture 141 and angled passage 178 with an axis Y that is angularly offset by an angle α from the longitudinal axis X of the catheter insertion device 100, which may be between 3 to 10 degrees, such as 8 degrees. The aperture 141 may have a diameter between 0.020 to 0.050 inches, such as 0.035 inches. As a result, the stabilizer 140 produces a drag force on the drag section 154 of the guidewire 150 by virtue of the guidewire 150 interfering with the inner surface of the angled passaged 178 and the aperture 141 as the outer body 130 is moved from the first position P1 to the second position P2. Moreover, the stabilizer 140 (including angled passage 178), axis Y, and axis X, may exist in a vertical plane parallel with channel 133 and guidewire advancer 137, such that the stabilizer 140 is symmetrical along the vertical plane defined by line A—A as seen in FIG. 7. This configuration also provides a partial seal by virtue of this interference between the stabilizer 140 and the drag section 154. Thus, this configuration provides a clearance that is sufficient to routinely prevent blood flow from the inner body 110 chamber of the catheter insertion device 100, while still allowing air to pass through.

The stabilizer 140 may also have a proximal upper surface 145 configured to abut with a first internal surface 161 of the outer body 130 and a distal upper surface 146 configured to be inserted into proximal end 112 of the inner body 110 which abuts with a second internal surface 162 of the outer body 130. At the second internal surface 162, the outer body 130 may include a strengthened bridge portion 164 to provide stability during attachment or detachment between the inner body 110 and the outer body 130. Moreover, this configuration prevents the inner body 110 from bowing or deforming when the user applies a threshold force against the outer body 130 in a proximal direction.

To achieve this, the distance d1 between the proximal upper surface 145 and the first internal surface 161 may be less than a deflection distance d2 when the threshold force is applied to the finger 182. The distance d3 between the distal upper surface 146 and the second internal surface 162 may be less than the deflection distance d2 when the threshold force is applied to the finger 182. In implementations of the disclosure, the deflection distance d2 may be between 0.005 to 0.030 inches, such as 0.015 inches depending on materials and geometry. Moreover, the distance d1 and distance d3 may be equa1 to each other and may be less than 0.030 inches, for example 0.005 inches, or distance d1 and distance d3 may be equal 0 inches in which case the proximal upper surface 145 may abut the first internal surface 161 and the distal upper surface 146 may abut the second internal surface 162.

Further, in another aspect of the present invention, a plane formed by the longitudinal axis X of the catheter insertion device, angle α and the axis Y may be substantially orthogonal with a center line of the channel 133, the projection 142, and the bowing prevention surfaces 161, 162 of the outer body 130 to simplify the manufacturing of the catheterization device 100.

Additionally, the stop member 135 provides for an override mechanism 180 configured to provide a hard stop against the projection 142 of the stabilizer 140 for preventing the guidewire 150 from being retracted past a predetermined position, while also allowing the user to detach outer body 130 from the inner body 110. For instance, when the outer body 130 is in the fully retracted position P1, the projection 142 of the stabilizer 140 abuts a flexible finger 182 that provides a hard stop against the stabilizer 140, thus preventing the outer body 130 from being retracted any further. Moreover, this finger 182 results in a terminus of the elongated channel 133. In this position, the projection 142 is nested in a bend of the flexible finger 182. The portion of the flexible finger 182 also includes an angled surface configured to allow the projection 142 to cause the flexible finger 182 to deflect downward when a sufficient amount of force is applied.

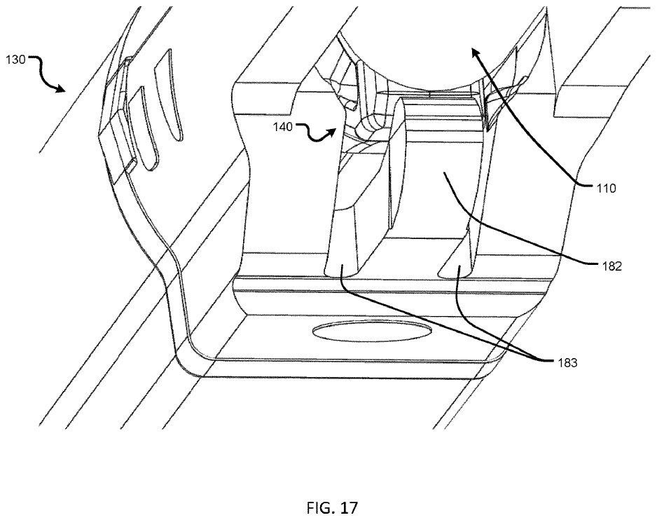

As seen in FIG. 17, the resiliency of the flexible finger 182 is in part due to the cantilevered nature of the structure of the finger 182 aided by a plurality of cutouts 183 from the outer body 130 to provide the elasticity necessary for the finger 182 to deflect downward. The projection 142 deflects the flexible finger 182 down as it passes, thus allowing a user to detach the outer body 130 from the inner body 110 and the flashback chamber 114. Detaching the outer body 130 from the flashback chamber 114 may be desirable if the clinician wants to remove the guidewire 150 from the flashback chamber 114, i.e., during an occasional clinical technique used to mitigate an insertion that initially failed. In this way, the projection 142 of the stabilizer 140 may be configured to engage the finger 182 and push the finger 182 when a threshold force is applied against by the user from pulling the outer body 130 in a proximal direction. For example, the threshold force may be between 3N and 10N to provide for a tactile feel to the user.

In this way, the finger 182 and the projection 142 may be configured to allow the outer body 130 to detach from and/or attach to the inner body 110. Thus, this configuration will provide for a stop 135 when the user moves the outer body 130 from the second position P2 to the first position P1. As such, when a user pulls the outer body 130 further out from this position, the finger 182 will deflect and elastically deform to allow enough of a distance for the outer body 130 to release from the projection 142 of the stabilizer 140; and thus the outer body 130 may be removed from the inner body 110.

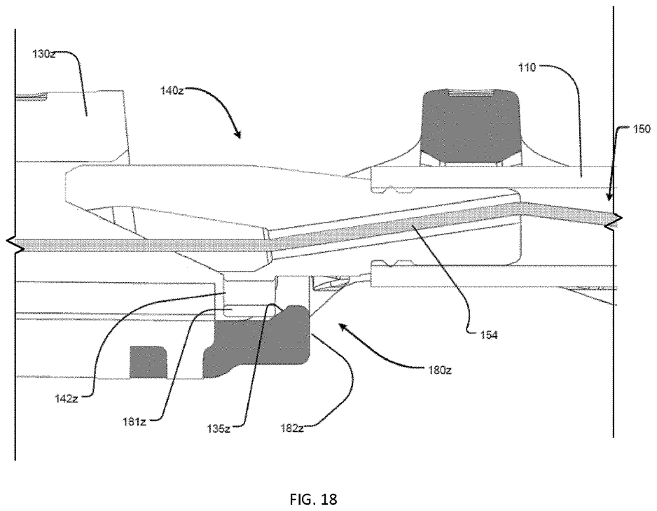

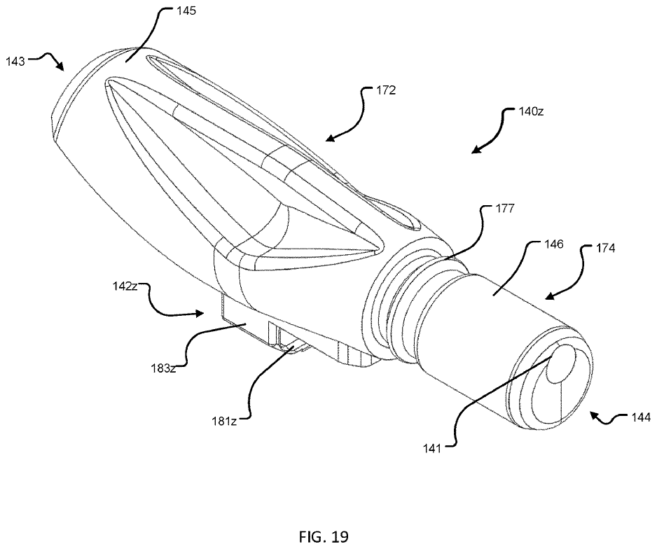

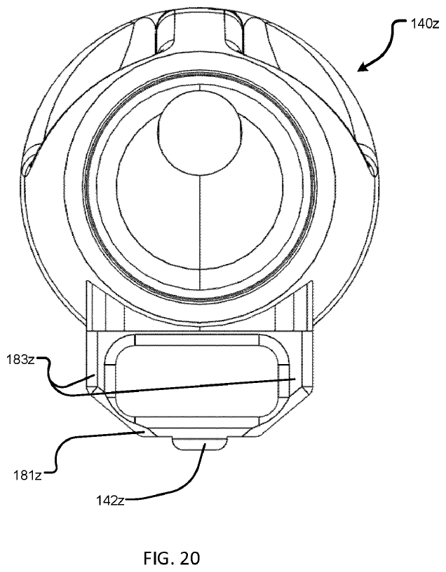

FIGS. 18-20 illustrates a stabilizer 140z and override mechanism 180z according to another aspect of the disclosure. The catheter insertion device 100 includes all the features described in FIGS. 1-17 except the projection or rudder 142z also includes a flexible or compressible bottom portion 181z and the outer body 130z has a rigid distal surface 182z rather than a flexible finger. Similarly, in this aspect, the stop member 135z also provides for an override mechanism 180z configured to provide a hard stop against the projection 142z of the stabilizer 140z for preventing the guidewire 150 from being retracted past a predetermined position, while also allowing the user to detach outer body 130z from the inner body 110.

However, in this aspect, when the outer body 130z is in the fully retracted position P1, the projection 142z of the stabilizer 140z abuts a rigid distal surface 182z of the outer body 130z that provides a hard stop against the stabilizer 140z, thus preventing the outer body 130z from being retracted any further. Moreover, this rigid distal surface 182z also results in a terminus of the elongated channel 133z. In this position, the projection 142z is nested in a bend of rigid distal surface 182z.

The portion of the rigid distal surface 182z also includes an angled surface configured to allow the projection 142z to elastically deform or compress its bottom portion 181z to deflect upward when a sufficient amount of force is applied. The bottom portion 181z deflects up as it passes over the rigid distal surface 182z, thus allowing a user to detach the outer body 130z from the inner body 110 and the flashback chamber 114.

The bottom portion 181z may also include a pair of side walls 183z configured to flex and bend inward or outward to deflect the bottom portion 181z upward. Detaching the outer body 130z from the flashback chamber 114 may be desirable if the clinician wants to remove the guidewire 150 from the flashback chamber 114, i.e., during an occasional clinical technique used to mitigate an insertion that initially failed. In this way, the projection 142z of the stabilizer 140z may be configured to engage the rigid distal surface 182z and compress the bottom surface 181z when a threshold force is applied against by the user from pulling the outer body 130z in a proximal direction. For example, the threshold force may be between 3N and 10N to provide for a tactile feel to the user.

FIG. 21 illustrates the proximal section 155 of the guidewire 150. In this section, diameter may be the same uniform thickness as the drag section 154 of the guidewire 150, such as 0.0165 inches in diameter. The guidewire 150 may have a curved proximal section 155 in order to facilitate attachment to the proximal end of the outer body 132. This configuration allows for the outer body 130 and the guidewire 150 to be able to move together as one unit with respect to the inner body 110.

Moreover, an anchor hole 163 may be provided on the proximal end of the outer body 132. The proximal section of the guidewire 155 may be received through the anchor hole 163 and bent or crimped so as to fixedly secure the guidewire 150 to the outer body 130. In some implementations, the proximal end of the guidewire 155 may be attached within a receptacle 164 formed at the proximal end of the outer body 132. In other implementations, the proximal section 155 may be attached to the proximal end of the outer body 132, such as by swaging or welding the end of the guidewire 150. Adhesive such as glue or other bonding material may also be used to further fixedly secure the proximal end of the guidewire to the handle. The location of the anchor hole 163 may be axially aligned with the location of the proximal opening of the angled passage 178 at the proximal end of the stabilizer to ensure the spring force between the guidewire 150 and the stabilizer 140 remains constant or only slightly increases as the guidewire 150 bends when passing through the angled passage 178 of the aperture 141 of the stabilizer 140.

The geometry of the angled passage 178 provides the proper amount of force (i.e., spring force) between the guidewire 150 and the stabilizer 140 so that there is a sufficient amount of frictional resistance between the guidewire 150 and the stabilizer 140 in order to provide drag to the guidewire advancer 137 to keep it from unintentionally sliding relative to the flashback chamber 114 and the inner body 110, e.g., due to gravity. The proper amount of force between the guidewire 150 and the stabilizer 140 further allows a user to easily and smoothly slide the guidewire actuator 137 over the outer body 130. This is controlled by a combination of size of the hole, angle of the through hole, length of the through hole, width of the through hole, and materials and surface finish of the stabilizer 140 and the guidewire 150. The degree and shape of how the wire bends then is just however the wire naturally is shaped from being passed through the angled passage 178.

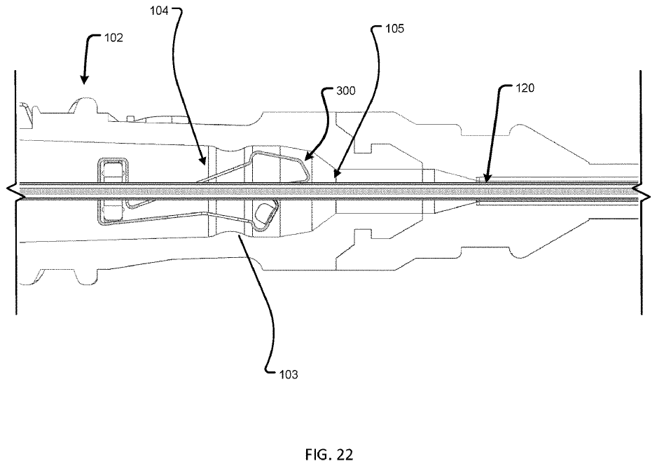

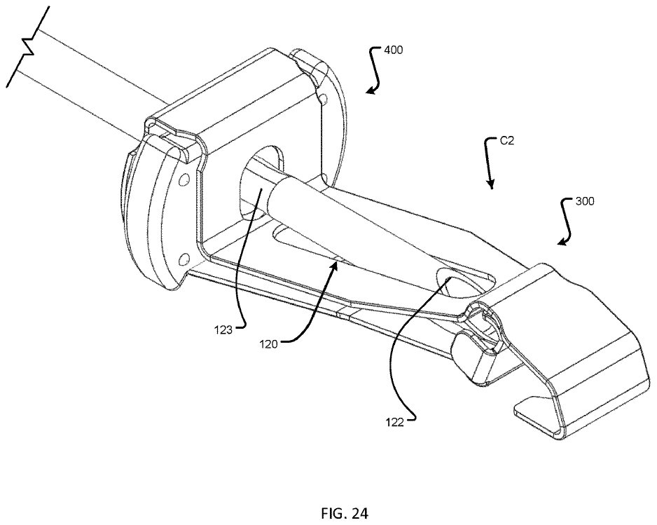

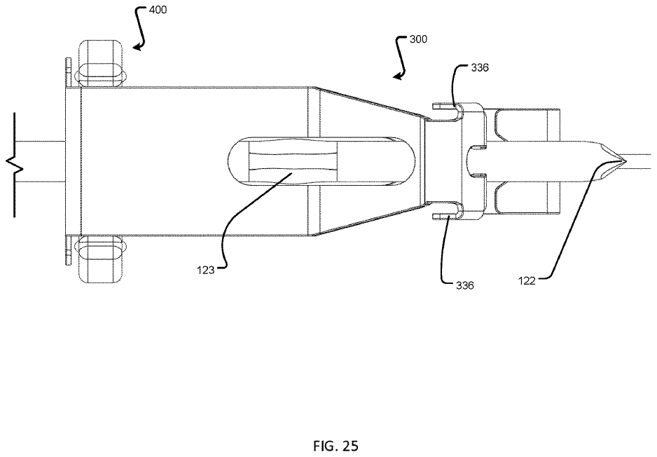

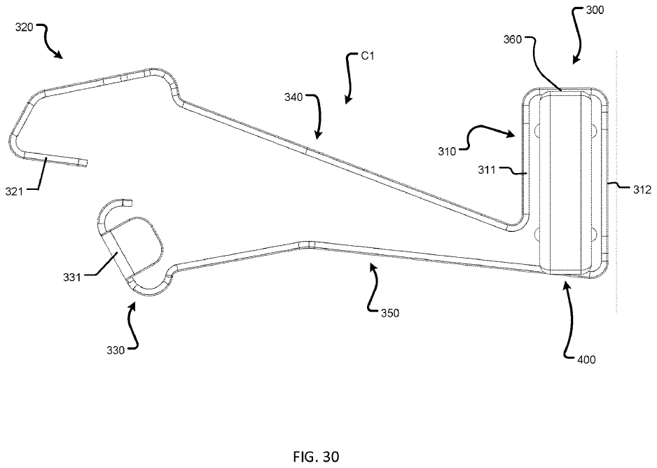

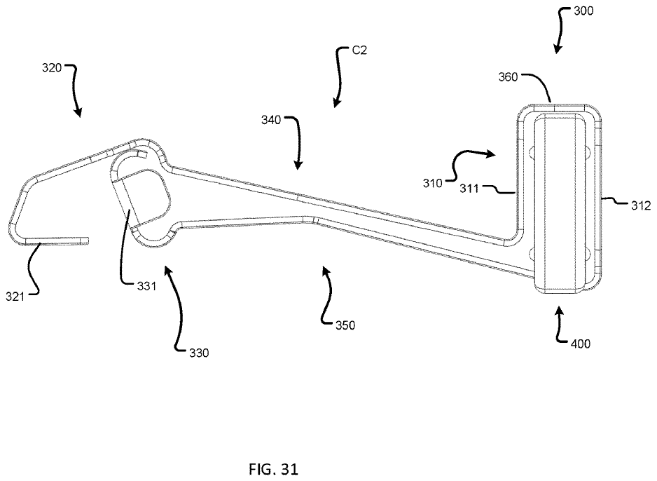

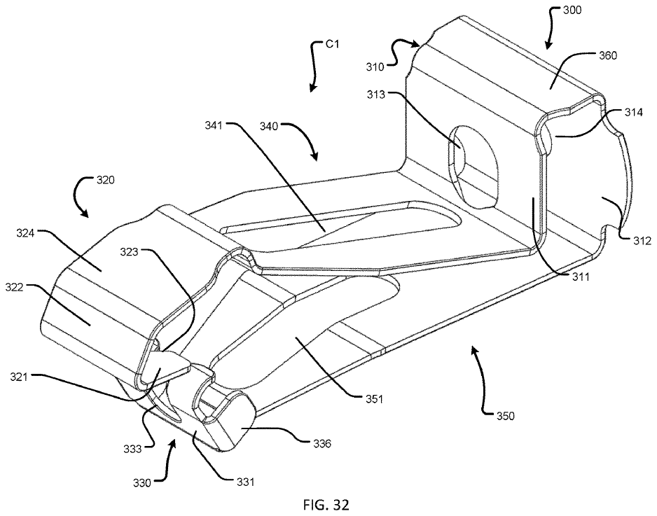

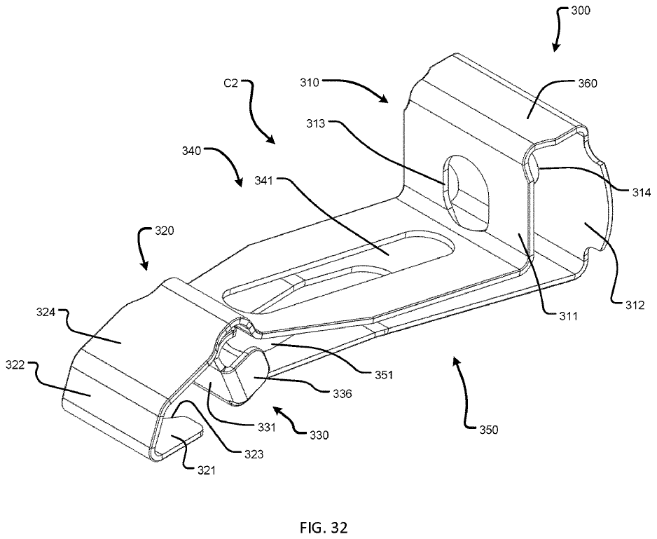

Referring to FIGS. 22-33, another aspect of the present invention relates to a safety clip 300 of the catheter insertion device 100. The safety clip 300 may include a proximal portion 310 with a first proximal wall 311 directly connected to a second proximal wall 312 at a proximal clip bend 360. This proximal clip bend 360 may have a single curve, may be rectilinear, or may otherwise be any shape known to those skilled in the art capable of providing an elastic force. The first proximal wall 311 may have a first proximal aperture 313 and the second proximal wall 312 may have a second proximal aperture 314. Further, the second proximal wall 312 may be located more proximal than the first proximal wall 311. In this manner, the needle 120 may slide through the first proximal aperture 313 and the second proximal aperture 314.

Additionally, the safety clip 300 may have a first distal portion 320 with a first distal wall 321, which may have a first distal slot 323 for receiving the needle 120. The first distal wall 321 may be connected to a first distal face 322 by a second distal face 324. The safety clip 300 may also have a second distal portion 330 having a second distal wall 331 connected to a first distal face 332 by a second distal face 334. Further, a first distal slot 323 may extend through a portion of the first distal wall 321, the first distal slot 323 configured to receive a guidewire of the needle. The first distal wall 321 and/or the second distal wall 331 may have lateral covers to further aid in sharps protection. For example, as seen in the Figures, the second distal wall 331 may have a pair of lateral covers 336.

Moreover, the safety clip 300 may have a second distal portion 330 with a second distal wall 331, the second distal wall 331 having a second distal slot 333 for receiving the needle 120. There may also be a second distal slot 333 extending through a portion of the second distal wall 331 and the first distal face 332, which may be configured to receive the guidewire 150 of the needle 120. The first distal wall 321 and the second distal wall 331 may have various shapes. For example, the walls may be angular or flat such as the first distal wall 321.

The safety clip 300 may also include a first flexible clip arm 340 which may extend from the first distal portion 320 to the first proximal wall 311 and may include a first elongated medial slot 341 for receiving the needle 120. The safety clip 300 also can include a second flexible clip arm 350 extending from the second distal portion 330 to the second proximal wall 312, where the first flexible clip arm 340 and the second flexible clip arm 350 contacts the needle 120. The second clip arm 350 may also include a second elongated medial slot 351 configured to laterally receive a second portion of the needle.

This design allows for the safety clip 300 to elastically move between two positions. A first, open configuration C1, and a second, closed configuration C2. It is important to note that, at rest, the safety clip 300 is configured so the first clip arm 340 and the second clip arm 350 are biased toward the closed configuration C2. Thus, the first clip arm 340 and/or the second clip arm 350 may be configured to press against the needle when in the closed configuration C2. In this way, the first clip arm 340, the second clip arm 350, or both may be configured to press against the needle when in the closed configuration C2. Additionally, the first proximal aperture 313 may be coaxial with the second proximal aperture 314 and the first distal face 322 and the first distal face 332 may be configured to press against the needle when the first clip arm 340 and the second clip arm 350 are in the open configuration. Furthermore, the second distal face 334 may be configured to abut the first distal wall 321 when the first clip arm 340 and the second clip arm 350 in the closed configuration. Moreover, a width of the first distal slot 323 may be greater than a width of the first medial slot, and a width of the second distal slot 333 may be greater than a width of the second medial slot.

As a result, the first flexible clip arm 340 and the second flexible clip arm 350 may be configured to flex between the first, open configuration C1 and the second, closed configuration C2 to move the safety clip 300 relative to the needle. Further, the first flexible clip arm 340 and the second flexible clip arm 350 are configured to move between the open configuration C1 and the closed configuration C2 as the first flexible clip arm 340 and the second flexible clip arm 350 are biased toward the closed configuration C2 in the rest state of the safety clip 300. Additionally, the first flexible clip arm 340 and the second flexible clip arm 350 are able to move to the closed configuration C2 with or without the guidewire being advanced beyond the tip of the needle. In some implementations, the safety clip 300 may be configured to move to an intermediate position in which the first flexible arm 340 is moved to a partially closed configuration, due to the guidewire being advanced, and the second flexible arm 350 is moved to the closed configuration as described herein.

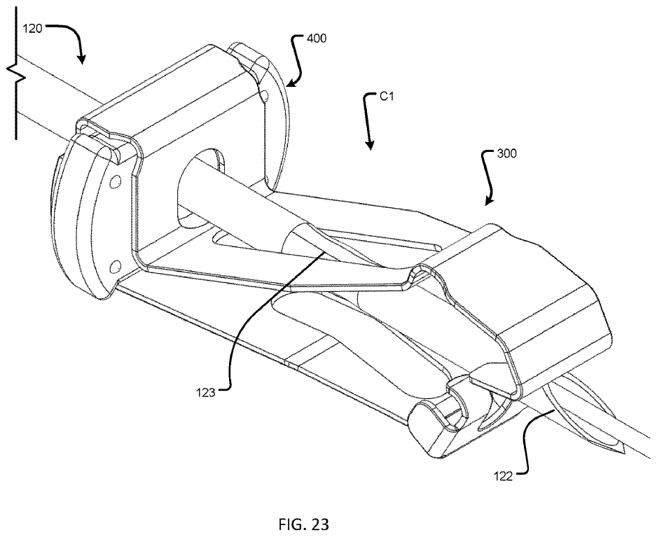

This flexing movement occurs when the first distal wall 321 and the second distal wall 331 of the safety clip 300 reach the needle tip and the first proximal aperture 313 reaches a swaged portion of the needle 120. Best seen in FIG. 23 this swaged portion 123 is configured to be in an oval shape and prevents the safety clip 300 from being pulled easily off the needle tip as the second proximal aperture 314 of the safety clip 300 has a smaller diameter than the major outer diameter of the swaged portion.

For example, when the first flexible clip arm 340 and the second flexible clip arm 350 are in the open configuration C1, the safety clip 300 may be able to slide along a length of the needle 120 and the first distal wall 321 and the second distal wall 331 are spaced apart. Additionally, when the first flexible clip arm 340 and the second flexible clip arm 350 are in the closed configuration C2, the second proximal aperture 314 may be configured to engage a swaged portion 123 of the needle. Further, in the closed configuration C2, the first distal wall 321 and the second distal wall 331 overlap to cover the tip 122 of the needle 120 as the first flexible clip arm 340 and the second flexible clip arm 350 move to the rest state of the safety clip 300 in the second configuration C2. Moreover, the first distal slot 323 and the second distal slot 333 may be configured to allow the first flexible clip arm 340 and the second flexible clip arm 350 to move to the closed configuration C2 with a guidewire advanced beyond the tip of the needle and may allow a guidewire to be retracted while in the closed configuration C2.

Thus, due to the first flexible clip arm 340 and the second flexible clip arm 350 being biased toward the closed configuration C2, the first distal wall 321 and the second distal wall 331 may be configured to cover a needle tip 122 to protect the user from injury. As seen in the Figures, to form such a cover, the first distal wall 321 is more distal than the second distal wall 331. However, in other aspects of the invention, the second distal wall 331 may be more distal than the first distal wall 321. As seen in the Figures, the first flexible clip arm 340 and the second flexible clip arm 350 do not cross each other in either the open configuration C1 or the closed configuration C2.

Accordingly, this design allows the safety clip 300 to easily be made in one piece. The safety clip 300 may be made by any known manufacturing method known to those skilled in the art such as 3D printing, casting, forging, die forming, and/or the like. Additionally, the clip may be made of any flexible, resilient or elastic material, such as a metal including aluminum or stainless steel, or plastic, capable of providing enough elastic force for the safety clip 300 to move between the open configuration C1 and the closed configuration C2. For example, the safety clip 300 may be manufactured of a single piece of metal, stamped, and then bent into shape.

To prevent a catheter 101 from being removed inadvertently from the catheter insertion device 100 before the safety clip 300 covers the needle tip 122 in its closed configuration C2, the catheter hub 102 may include a protrusion or protrusion 103. This protrusion 103 is configured so that the internal diameter of the catheter hub 102 interferes with the safety clip 300 when the safety clip 300 is in its open configuration C1 and does not interfere with the safety clip 300 when the safety clip 300 is in its closed configuration C2. This feature also allows the catheter 101 to move simultaneously with the safety clip 300 when the user inserts the catheter 101 into the vessel 200 of the patient. As such, the protrusion 103 may define the hub 102 into a proximal cavity and a distal cavity.

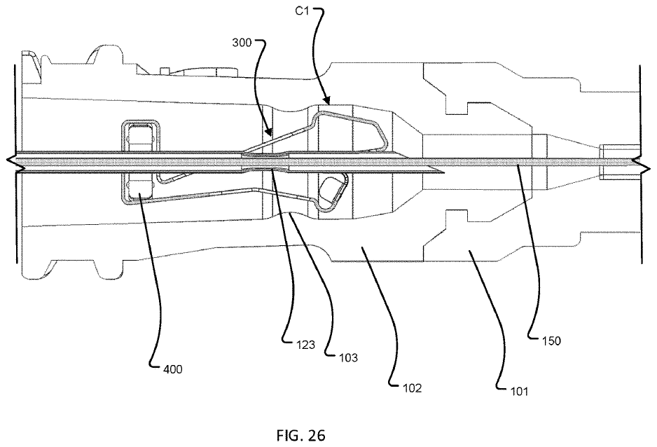

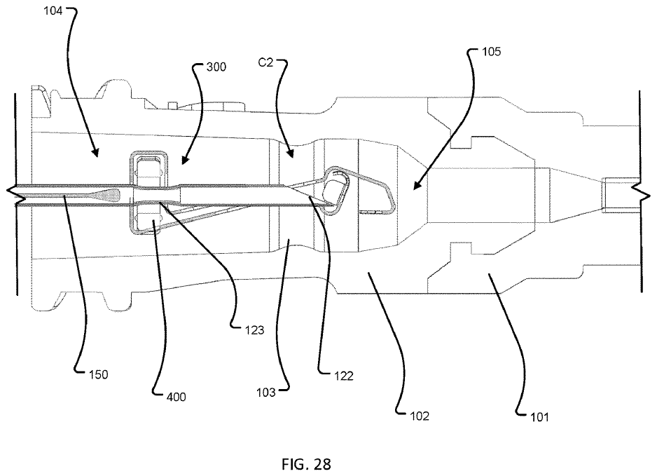

That is, in FIG. 26, the safety clip 300 is in the open configuration C1 where its first distal wall 321 and the second distal wall 331 are spread apart at a radial distance beyond the internal diameter of the catheter hub 102 at the protrusion 103. When the safety clip 300 reaches the end of the needle 120, in FIG. 28, the needle 120 flexes to the closed configuration C2 compressing the first distal wall 321 and the second distal wall 331 together in such a way to allow the catheter 101 to freely move with respect to the needle 120, the safety clip 300, and the medical device. Thus, as the catheter 101 is inserted into the vessel 200 of the patient the safety clip 300 will also cover the needle tip to protect the user from injury.

Additionally, the safety clip 300 may include a one-piece body. Moreover, the first distal portion 320 of the safety clip 300 may include a first curved elbow 325 connecting the first clip arm 340 to the first distal wall 321, and the second distal portion 330 of the safety clip 300 may include a second curved elbow 335 connecting the second clip arm 350 to the second distal wall 331. Further, the first and second curved elbows may be configured to contact a bump 103 portion within a hub of the catheter 101 to prevent removal of the safety clip 300 from inside the hub when in the open configuration. The safety clip 300 may also be configured to be removed from the hub of the catheter 101 when the first clip arm 340 and the second clip arm 350 are in the closed configuration.

Referring to FIGS. 22-33, in another aspect of the invention, the safety clip 300 may include a flexible keeper 400 made of silicone or another elastomeric material between the first proximal wall 311 and the second proximal wall 312. The operation of the safety clip 300 and the structure of the safety clip 300 with and without the keeper 400 are substantially the same. However, the keeper 400 may be configured to ensure the safety clip 300 remains properly retained within the catheter hub 102 as the catheter 101 is inserted into the vessel 200 of the patient and removed from the catheter insertion device 100. The keeper 400 may thus be configured to provide frictional resistance against an inner surface of the catheter hub 102 to prevent the safety clip 300 from pulling out of the hub 102 absent application of a threshold force.

For this purpose, the catheter hub 102 initially has a larger inner diameter than the outer diameter of the keeper 400, but the inner diameter of the catheter hub 102 slowly decreases in diameter as the keeper 400 is pushed into the catheter hub 102 until the outer diameter of the keeper 400 is slightly larger than the inner diameter of the catheter hub 102. This arrangement creates friction or drag between the keeper 400 and the catheter hub 102 to help ensure the safety clip 300 stays with the catheter 101 until the safety clip 300 can activate towards is closed configuration C2 to cover the needle tip 122 as the catheter 101 is being inserted into a vessel of a patient and the medical device is being removed from the catheter 101.

The keeper 400 may be of one piece that fits within the space between the first proximal wall 311 and the second proximal wall 312 of the safety clip 300 as seen in FIG. 23 or could include projections integral with the safety clip 300 extending from the first proximal wall 311 and/or second proximal wall 312 of the safety clip 300. It is important to note that the force to pull the swaged portion 125 of the needle 120 through the internal diameter of the second proximal aperture 314 is much greater than the force to pull the keeper 400 from the internal diameter of the catheter 101.

The keeper 400 may also include a through-hole configured to slidably receive the needle. Further, a diameter of the through-hole may be greater than a diameter of the first proximal aperture 313 and a diameter of the second proximal aperture 314. Additionally, in embodiments where the safety clip 300 uses a keeper 400, the friction or drag force of the keeper 400 should not be large enough so that it would pull on the catheter 101 during removal of the medical device after the catheter 101 has been fully inserted into the blood vessel 200 of the patient. However, a person skilled in the art would be able to determine through routine experimentation the desired interference fit and material capable of creating the desired results of the keeper 400 as described herein.

To assemble the catheter insertion device 100 with the safety clip 300 the keeper 400 is pushed into a slot of the safety clip 300 then a tool is used to spread open the first clip arm 340 and the second clip arm 350. The back end of the needle 120 is then slid into the safety clip 300. In this way, the back end of the needle first slides past the first distal slot 323, then past the second distal slot 333, then through the first elongated medial slot 341, past the second elongated medial slot 351, through the first proximal aperture 313, and finally through the second proximal aperture 314 of the safety clip 300. The safety clip 300 will slide along the length of the needle 120 and will rest in the center of the length of the needle 120.

Here, the needle 120 and safety clip 300 and keeper 400 assembly is removed from the tool used to spread open the first clip arm 340 and the second clip arm 350. At this point, the needle 120 is glued, or otherwise affixed to the needle hub. A catheter 101 may then be slid over the needle tip 122 of the catheter insertion device 100 which causes the first curved elbow 325 and the second curved elbow 335 to contact the protrusion 103 of the catheter 101. Pushing on the catheter 101, slides the safety clip 300 along the needle 120 until the safety clip 300 contacts the needle hub 121. At this point, the needle hub 121 pushes back onto the safety clip 300 and causes the arms of the safety clip 300 to elastically compress until the first distal wall 321 and the second distal wall 331 have moved past the protrusion 103 at which point the safety clip 300 uncompresses back to its original geometry. Thereafter, the safety clip 300 will move simultaneously with the catheter 101 along the length of the needle 120 until the safety clip 300 activates. This structure of the safety clip 300 thus creates an over the needle catheter 101 that conceals the safety clip 300 during catheter insertion.

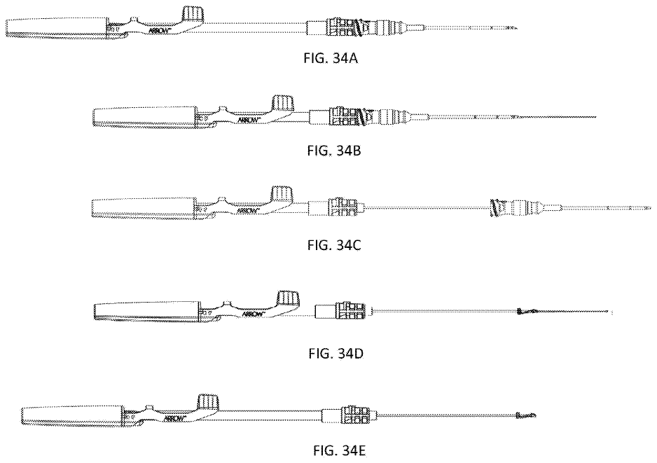

FIGS. 22-34E show the safety clip 300 in operation with the catheter insertion device 100. In particular, catheter insertion may begin with the catheter insertion device 100 in the first position P1 seen in FIG. 34A. Then, after preparing the patient for needle insertion, the user (e.g., practitioner or clinician) may insert the tip 122 of the needle 120 at approximately a 30° insertion angle (γ) with respect to the surface of the skin to access the vessel 200 of the patient as illustrated in FIG. 3. At this point, blood will flash back into the inner body 110 before the advancement of the guidewire 150. This blood flashback provides indication to the user that the needle 120 has accessed the blood vessel 200.

From here, the user moves the outer body 130 from the first position P1, where a guidewire tip 151 is within the needle 120, to a second position P2 as in FIG. 34B, where the guidewire tip 151 is advanced into the vessel 200 of the patient. This movement may be done with one hand by pushing continuously on the flag 137 at a push point. The guidewire tip 151 forms an atraumatic end and has sufficient elasticity to bend so when the guidewire tip 151 hits the back wall of the vessel 200, the guidewire 150 will bend from the 30° insertion angle and move substantially parallel into the artery to form a guide for the catheter 101.

At the same time, when the outer body 130 moves between the first position P1 and the second position P2, the outer body 130 slides over the inner body 110 and the guidewire 150 fixed to the proximal end of the outer body 132 simultaneously move through an inner portion 113 of the inner body 110. At this point, the guidewire 150 extend through an aperture 141 of a stabilizer 140 fixed to a proximal end of the inner body 112, and the stabilizer 140 is configured to stabilize the guidewire 150 when the outer body 130 moves from the first position P1 to the second position P2 by virtue of the aperture 141 having the axis Y angularly offset by angle α from the longitudinal axis X of the catheter insertion device 100.

From here, as seen in FIG. 34C, the user may advance the catheter 101 forward to insert it into the vessel by pushing the catheter hub 102 forward over the needle until the catheter hub 102 is fully advanced into the insertion site. As the user moves the catheter 101 forward, a safety clip 300 within the catheter 101 will slide along the length of the needle 120.

At this point, the needle 120 may be retracted to remove it from the catheter 101. As the needle 120 is retracted and pulled fully out of the catheter hub 102, the needle tip 122 will be retracted until the arms of the safety clip 300 are able to activate and the distal walls (321, 331) will begin to close around the needle tip 122 to protect the user from injury.

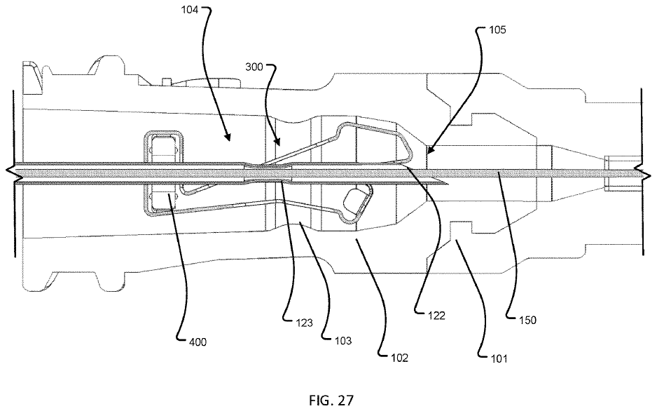

For example, when the first clip arm 340 and the second clip arm 350 are in the first configuration C1, the safety clip 300 may be able to slide along a length of the needle 120 and the first distal wall 321 and the first distal wall 331 are spaced apart. Further, when the first clip arm 340 and the second clip arm 350 are in the first configuration C1, the first curved elbow 325 and the second curved elbow 335 are engaged with the protrusion 103 of the catheter hub 102 as the catheter 101 is inserted into the vessel of the patient. When the user begins to retract the needle after catheterization, for example, after the catheter is fully advanced into the insertion site in the vessel 200, the needle will be moved proximally relative to the safety clip 300 to a point as seen in FIG. 34C. At this point, the safety clip 300 is still in the open configuration C1. However, as the user continues to retract the needle, the safety clip 300 will activate at which point, the first distal wall 321 and the second distal wall 331 are located approximately at the beginning of the beveled tip 122 of the needle, as seen in FIG. 27. Here, as the catheter has fully accessed the blood vessel 200 of the patient, the user may pull back the catheter insertion device 100 from the insertion site back as seen in FIG. 34D. As the user pulls the catheter insertion device 100 back, the first distal wall 321 and the second distal wall 331 of the safety clip 300 will slide down the beveled tip 122 of the needle and activate the first clip arm 340 and the second clip arm 350 to begin to close the safety clip 300 around the needle 120 as the needle is further retracted because both the first and second flexible clip arms 340, 350 are biased to move toward each other.

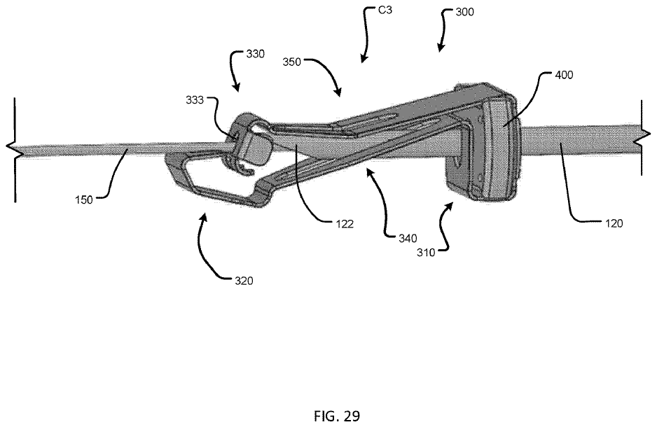

Thus, in some implementations, the guidewire 150 is not retracted when the needle 120 is removed. As seen in FIG. 29, the safety clip 300 may be configured to move to an intermediate configuration C3 in which the first flexible arm 340 is moved to a partially closed configuration and the second flexible arm 350 is moved to the fully closed configuration. Rather than moving fully to the closed configuration C2, as seen in FIG. 28, the safety clip 300 moves to the intermediate configuration C3 in FIG. 29 where the first distal slot 323 and the first distal face 322 abuts the guidewire 150. As the needle 120 is further retracted, the first distal face 322 slides along the surface of the guidewire 150. The guidewire 150 is made of a stiffer material than the safety clip 300 to prevent the guidewire from bending when the first distal face 322 abuts and slides along the guidewire 150 in this intermediate configuration C3. Moreover, when the needle 120 is retracted further, the beveled tip 122 of the needle clears the second distal portion 330 and moves to the fully closed configuration C2.

This is because the second distal slot 333 is configured to receive the guidewire 150 thus allowing the second flexible clip arm 350 to fully close, while the first distal slot 323 is configured to abut the surface of the guidewire 150 allowing the first flexible clip arm 340 to only partially close. In other words, the distal face 322 and/or a portion of the first distal slot 323 continues to abut against the guidewire which prevents the first flexible arm from being biased to its fully closed position. In this intermediate configuration C3, even though the guidewire 150 remains advanced past the first and second distal portions 320, 330 of the safety clip 300, the needle tip 122 remains proximal of both the first and second distal portions, and thus the safety clip 300 is able to provide sharps protection from the needle tip 122.

Further, in this intermediate configuration C3 of the safety clip 300, the guidewire 150 may still be retracted until the first distal face 322 no longer abuts the guidewire, at which point the first flexible arm 340 is biased toward the fully closed configuration C2. Accordingly, the safety clip 300 is configured to provide sharps protection to the needle tip 122 when the guidewire 150 is advanced past the first and second distal portions 320, 330 of the safety clip 300, and when the guidewire 150 is retracted from the first and second distal portions 320, 330 of the safety clip 300. Thus, after the needle 120 is fully removed from the catheter, the user may pull back the guidewire actuator 137 to retract the guidewire 150 and return the catheter insertion device 100 from the second position P2 to the second position P1, as seen in FIG. 34E.

However, even if the guidewire has been retracted prematurely, as the user retracts the needle 120, the first distal wall 321 and the second distal wall 331 of the safety clip 300 will slide down the beveled tip 122 of the needle and activate the first clip arm 340 and the second clip arm to move from the open configuration C1 to the closed configuration C2 as seen in FIG. 28. Thus, after the needle 120 and guidewire 150 is fully removed from the catheter, the first clip arm 340 and the second clip arm 350 will be in the second configuration C2. Additionally, when the first clip arm 340 and the second clip arm 350 are in the second configuration C2, the first proximal aperture 313 may cross over a swaged portion of the needle and the second proximal aperture 314 may then engage the swaged portion of the needle 120 such that the needle 120 is prevented from moving further. Thus, in this second configuration C2, the first distal wall 321 and the first distal wall 331 may be configured to cover a needle tip 122 to protect the user from injury. To form such a cover, the first distal wall 321 is more distal than the second distal wall 331. However, in other aspects of the invention, the second distal wall 331 may be more distal than the first distal wall 321. The first clip arm 340 and the second clip arm 350 do not intersect in either the first configuration C1 or the second configuration C2.

Moreover, as seen in FIG. 28, when the device is in the closed configuration C2, the first curved elbow 325 and the second curved elbow 335 will also clear the protrusion 103 of the catheter hub 102. At this point, the catheter insertion device 100 and guidewire 150 assembly are ready for removal. The user may also continue to pull back on the catheter insertion device 100. Because the safety clip 300 has activated, the needle 120 and the safety clip 300 are free to be removed from the catheter 101 with the safety clip fully in the closed configuration C2 and covering the tip of the needle.

In embodiments with a safety clip 300 including a keeper 400, pulling back on the catheter insertion device 100 will cause a slight pop to be felt as the keeper 400 is released due to the interference between the keeper 400 and the catheter hub 102. The user may continue to freely pull back the catheter insertion device from the catheter 101 until the needle 120 with its tip 151 covered by the safety clip 300 is entirely removed. At this point, the medical device may be discarded into a sharps bin for disposal, while the catheter 101 is left in the vessel 200 of the patient for treatment.

Additionally, when the first clip arm 340 and the second clip arm 350 are in the first configuration C1, the safety clip 300 may exert a first force on the needle 120 tending to return the safety clip 300 back to the second configuration C2. This feature of the safety clip 300 ensures that the safety clip 300 has a resting position intended to cover the needle tip to prevent injury and intended to be disengaged from the catheter hub 102. In contrast, the first configuration C1 ensures the safety clip 300 will actively hold onto the catheter hub 102 while tending to want to move towards the second configuration C2 once the safety clip 300 activates over the needle tip 122.

In one aspect of the disclosure, the inner body 110 may be made from medical grade polyurethane, polyvinylchloride (PVC), and/or other materials known to those skilled in the art. In some implementations, the inner body 110 may include indicia to indicate where the guidewire 150 is positioned in relation to the needle tip. For example, indicia of “0” indicates the needle tip is located at the needle tip, while indicia of “1” indicates the needle tip is located 1 unit past the needle tip. In aspects of the invention, these units may be a centimeter, an inch, or another preferred measurement known to those skilled in the art. In an aspect of the invention, the inner body 110 may extend from 3.5 inches to 6 inches in length, for example. However, a longer length is preferred because a longer length reduces the rate at which the inner portion 113 of the inner body 110 may fill with blood during continuous blood flashback.

In one aspect of the disclosure, the outer body 130 may be over-molded from clear polycarbonate and/or other materials known to those skilled in the art. This design allows the user to see the pulsatile blood return into the inner portion 113 of the inner body 110 as the outer body 130 moves from the first position P1 to the second position P2. The flag 137 of the outer body 130 may be made of high-density polyethylene (HDPE) and/or other materials known to those skilled in the art. The flag 137 may be black, blue, green, or any other color to contrast the flag 137 from the rest of the outer body 130 for easy user manipulation.

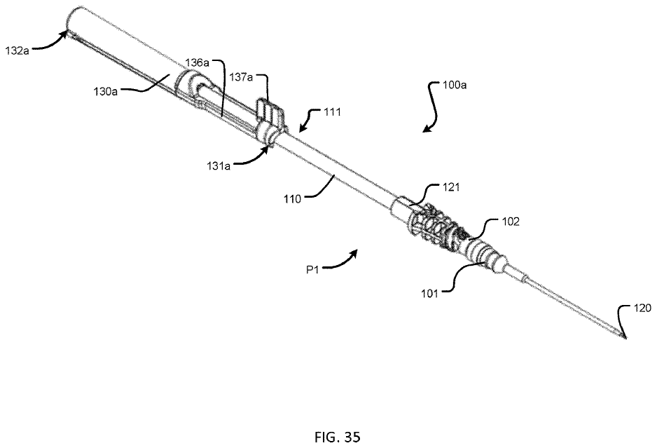

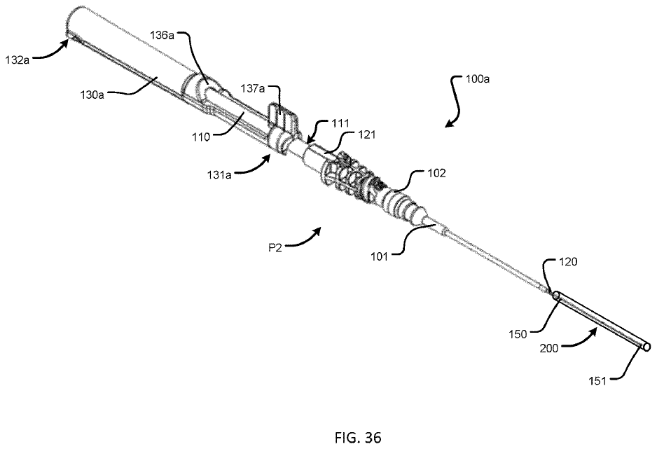

Referring to FIGS. 35 and 36, another implementation of the invention relates to a catheter insertion device 100a, like those seen and described in FIGS. 1-33E, with an inner body 110, a pulsatile blood flashback chamber 114, a hollow introducer needle 120 with a needle tip 122, a needle hub 121, and an outer body 130a configured to move between a first position P1 and a second position P2 as similarly otherwise described herein. Additionally, the catheter insertion device 100 may include a catheter 101, a catheter hub 102, and a guidewire 150 with a tip 151.

In an aspect of the invention, the catheter insertion device 100a may include a stabilizer 140a having an aperture 141a. The aperture 141a preferably may be round, but, square shaped apertures, elliptical shaped apertures, or other shaped apertures understood by those skilled in the art are contemplated. Additionally, the stabilizer 140a may be attached to a proximal end of the inner body 112.

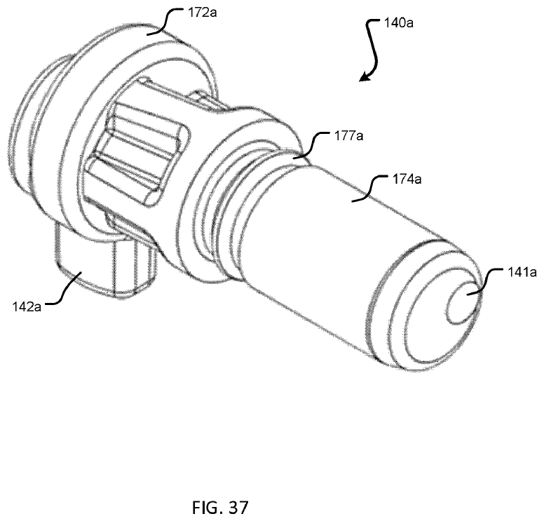

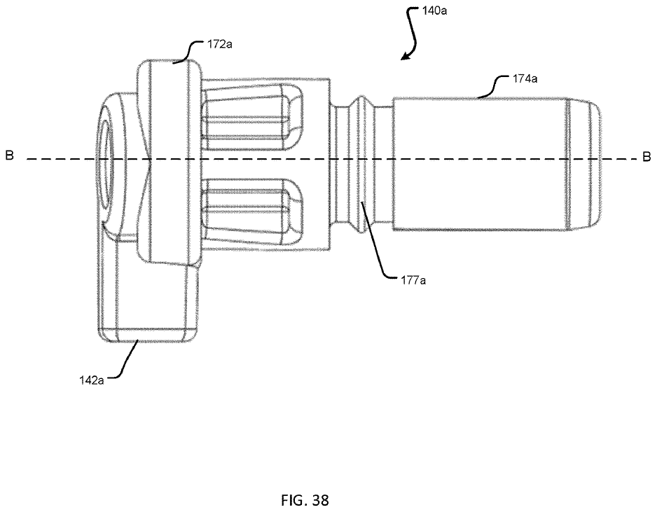

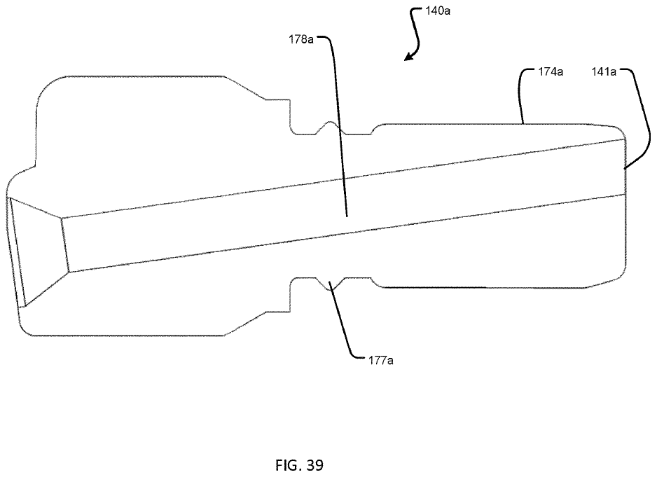

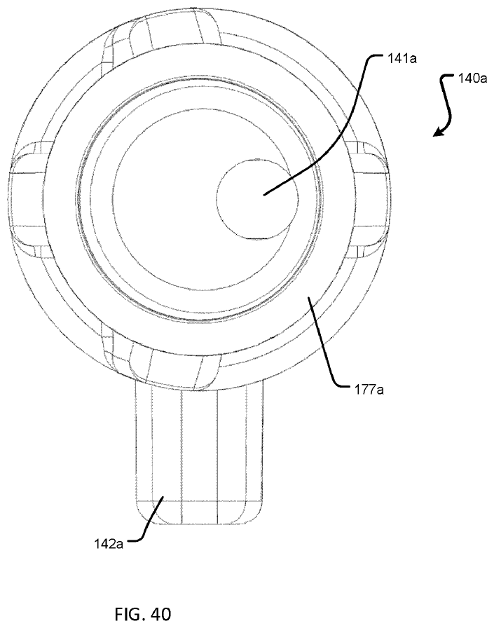

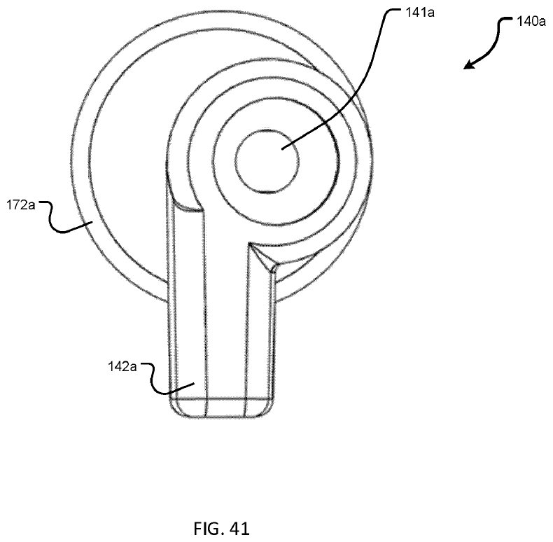

Referring to FIGS. 37-41, the stabilizer 140a of the catheter insertion device 100 may comprise a projection or rudder 142a, the outer body 130a may comprise an elongated slot 133a, and the rudder 142a may be configured to slide within the slot 133a to prevent or minimize the rotation of the outer body relative to the inner body when the outer body 130 moves between the first position P1 and the second position P2. The outer body 130a of the catheter insertion device 100 may further comprise a stop member 135a at a distal end of the slot 133a, located at a distal end of the outer body 130a. The stop member 135a may be configured to abut against the rudder 142a of the stabilizer 140a to prevent the outer body 130a from moving in a proximal direction relative to the inner body 110.

Moreover, the stop member 135a may be configured to provide a hard stop against the stabilizer 140a when the guidewire 150 is fully retracted. For instance, the stop member 135a may be configured to abut the rudder 142a during retraction of the outer body 130.

Similarly here, the stabilizer 140a may include a base 172a, and a distal portion of the stabilizer 140a includes a nose 174a. The rudder 142a may extend from the base 172a. An annular protrusion 177a is disposed between the base 172a and the nose 174a. The nose 174a and the annular protrusion 177a are configured to be received within the proximal end of the flashback chamber 114 of the inner body 110. The annular protrusion 177a may be configured to provide an interference fit with an inner surface of the flashback chamber 114 for securing the stabilizer 140a thereto. The aperture 141a of the stabilizer may further comprise an angled passage 178a extending from the proximal portion to the distal portion such that the angled passage 178a extends through the nose 174a and the base 172a. A proximal portion of the angled passage 178a may have a funnel shaped opening.

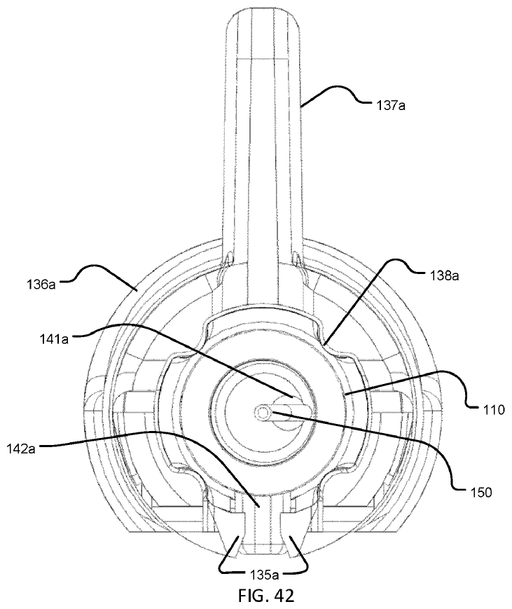

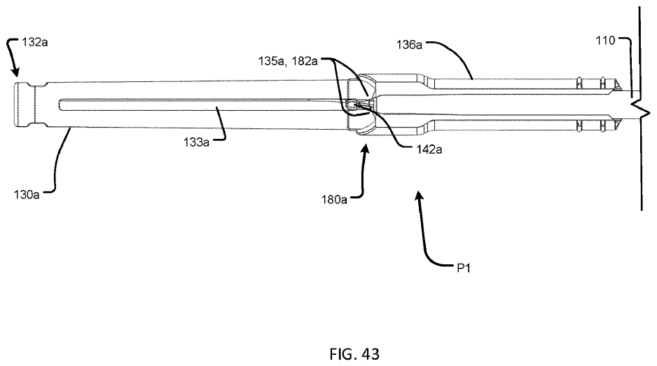

Additionally, as seen in FIGS. 42 and 43, the stop member 135a provides for an override mechanism 180a configured to provide a hard stop against the rudder 142a of the stabilizer 140a for preventing the guidewire 150 from being retracted past a predetermined position, while also allowing the user to detach outer body 130 from the inner body 110. For instance, when the outer body 130 is in the fully retracted position P1, the rudder 142a of the stabilizer 140a abuts a pair of flexible arms 182a that provide a hard stop against the stabilizer 140a, thus preventing the outer body 130a from being retracted any further. In this position, the rudder 142a is nested in a respective notch 184 formed on each flexible arm 182a. The portion of each flexible arm forming the respective notch also includes an angled surface configured to allow the rudder to enter a gap between the opposing flexible arms when a sufficient amount of force is applied. The rudder 142a spreads the opposing flexible arms 182a apart as it enters and passes through the gap, thus allowing a user to detach the outer body 130a from the inner body 110 and the flashback chamber 114. Detaching the outer body 130a from the flashback chamber 114 may be desirable if the clinician wants to remove the guidewire 150 from the flashback chamber 114, i.e., during an occasional clinical technique used to mitigate an insertion that initially failed. In this way, the rudder 142a of the stabilizer 140a may be configured to pass through the passage of the slot 133a when a threshold force is applied against the pair of flexible arms 182a. For example, the threshold force may be between 3N and 10N to provide for a tactile feel to the user.

In this way, these protrusions may be configured to allow the outer body 130a to detach from and/or attach to the inner body 110. Thus, this configuration will provide for a stop when the user moves the outer body 130a to the second position P2. As such, when a user pulls the outer body 130a further out from this position, each protrusion of the stop member 135a will elastically deform orthogonally to the slot 133a to allow enough of a gap for the outer body 130a to release from the rudder 142a of the stabilizer 140a. This aspect of the invention allows for ease of manufacturing and replacement of parts. By removing the outer body 130a from the inner body 110, the guidewire 150, and outer body 130a may be easily replaced if broken or damaged.

Further, the rudder 142a configured to slide along the slot 133a allows the catheter insertion device 100 to remain stable throughout the movement from the first position P1 to the second position P2. That is, the rudder 142a prevents the outer body 130a from rotating around the inner body 110 while moving between the first position P1 and the second position P2.

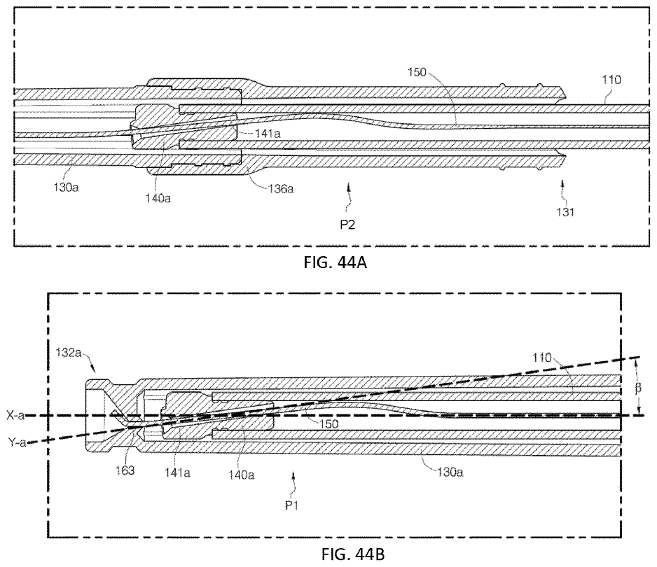

As seen in FIGS. 44A and 44B, an axis of the passageway 178a and aperture 141a may be positioned at an angle β, which may be between 3 to 10 degrees, such as 8 degrees, along the longitudinal axis of the catheterization device 100a. For example, the passageway 178a and aperture 141a may have the axis Y-a angularly offset by angle β from the longitudinal axis X-a of the catheter insertion device 100a. Preferably, angle β, axis Y-a and longitudinal axis X-a exist in a lateral plane (e.g., a plane parallel to the slot 133a) and are configured to keep the guidewire 150 away from the slot 133a and reduce risk of the guidewire 150 mistakenly bowing out of the slot 133a during use. In this way, directing the guidewire 150 from the slot 133a ensures that the guidewire 150 will not bow into the slot 133a if there is resistance that is encountered during an insertion procedure.

However, alternative embodiments of the stabilizer 140, 140a may also be implemented. For example, in another embodiment, the stabilizer does not include a rudder and the outer body does not include a slot, but rather the stabilizer is of a shape complimentary to the internal shape of the outer body, such as a square shape, to provide stability during movement from the first position P1 to the second position P2. Moreover, in another embodiment an elastomeric disk is provided to produce drag on the guidewire as a stiffening member, and the stabilizer aperture may be shaped to produce the drag. Other embodiments of a catheter insertion device envisioned by the disclosure may include all or portions of those as described in U.S. Provisional Application Nos. 63/779,990 and 63/780,101, herein incorporated by reference.

Additionally, as in FIG. 42, the outer body 130a of the catheter insertion device 100 may also comprise a guidewire actuator 137a on the distal end of the outer body configured to move the guidewire between the undeployed state as in the first position P1 and the deployed state as in the second position P2. A distal end of the guidewire actuator 137a may include a flag configured to allow a user to move the outer body between the first position and the second position. Further, a proximal end of the guidewire actuator 137a may be an over-molded portion 136a at a distal end of the outer body 130a. However, the guidewire actuator 137a may also be snap fit, press fit, welded, glued, or otherwise attached to the outer body 130a.

Here, the guidewire actuator 137a may include at least one projection extending inwardly from the outer body towards the inner body. Preferably the guidewire actuator 137a may include a plurality of projections 138a, such as the projections 138a seen in FIG. 42. This arrangement minimizes the contact points of the outer body 130a on the inner body to create a smooth operation of movement when the outer body moves between the first position and the second position and provides minimal friction due to the limited number of contact points created between each projection 138a and the outer surface of the flashback chamber 114.

Further, the arrangement between the stabilizer 140a, the angled passage 178a, and the aperture 141a, provides drag on the guidewire 150 as the outer body 130a moves between the first position P1 and the second position P2. This configuration also helps to create a tactile feel to the user. As such, the location of the guidewire actuator 137a is configured to create a stable push point allowing the user to easily move the outer body from the first position P1 and the second position P2 without buckling the guidewire 150. The guidewire actuator 137a further provides for an advancement grip and may be located at the distal end of the outer body or as close to the front of the device as possible to provide for shorter reach and enable the user to use the device with one hand.

While the catheter insertion device has been described in terms of what may be considered to be specific aspects, the present disclosure is not limited to the disclosed aspects. Additional modifications and improvements to the catheter insertion apparatus may be apparent to those skilled in the art. Moreover, the many features and advantages of the disclosure are apparent from the detailed specification, and thus, it is intended by the appended claims to cover all such features and advantages of the disclosure which fall within the spirit and scope of the disclosure.

Further, it is not desired to limit the disclosure to the exact construction and operation illustrated and described, and accordingly, all suitable modifications and equivalents may be resorted to, falling within the scope of the disclosure. Accordingly, the present disclosure should be considered as illustrative and not restrictive. As such, this disclosure is intended to cover various modifications and similar arrangements included within the spirit and scope of the claims, which should be accorded their broadest interpretation so as to encompass all such modifications and similar structures.

What is claimed is:

1. A catheter insertion device comprising:
an inner body defining a continuous pulsatile blood flashback chamber;
a needle hub having a proximal end attached to a distal end of the inner body;
a hollow introducer needle extending from a distal end of the needle hub, the introducer needle having a tip configured to pierce a wall of a blood vessel;
a catheter configured to slidably fit over the introducer needle, the catheter extending from a catheter hub;
an outer body configured to slidably receive a portion of the inner body;
a guidewire coupled to a proximal end of the outer body; and
a stabilizer coupled to a proximal end of the inner body, the stabilizer comprising a passage configured to receive the guidewire;
wherein the outer body is further configured to move between a first position in which the guidewire is at an undeployed state and a second position in which the guidewire is at a deployed state;
wherein a tip of the guidewire is located within the needle when the outer body is at the first position;
wherein the tip of the guidewire is advanced beyond the tip of the needle when the outer body is at the second position; and
wherein the passage of the stabilizer is configured to provide frictional resistance to the guidewire when the outer body moves between the first position and the second position.

2. The catheter insertion device of claim 1, wherein the passage is further configured to have an axis angularly offset by an angle with respect to a longitudinal axis of the catheter insertion device, wherein the angle is between 3 and 10 degrees.

3. The catheter insertion device of claim 1, wherein the stabilizer comprises a projection, the outer body comprises an elongated channel, and the projection is configured to slide within the elongated channel to prevent or minimize rotation of the outer body relative to the inner body when the outer body moves between the first position and the second position.

4. The catheter insertion device of claim 3, wherein the outer body further comprises a stop member at a distal end of the elongated channel configured to abut the projection of the stabilizer to prevent the outer body from moving proximally relative to the inner body.

5. The catheter insertion device of claim 4, wherein the stop member comprises a resilient finger forming a terminus of the elongated channel; and wherein the projection of the stabilizer is configured to elastically deflect the finger upon application of a threshold force on the longitudinal axis of the outer body to allow the outer body to detach from and/or attach to the inner body.

6. The catheter insertion device of claim 1, wherein the stabilizer comprises a rudder, the outer body comprises an elongated slot, and the rudder is configured to slide within the slot to prevent or minimize rotation of the outer body relative to the inner body when the outer body moves between the first position and the second position.

7. The catheter insertion device of claim 6, wherein the outer body further comprises a stop member at a distal end of the elongated slot configured to abut the rudder of the stabilizer to prevent the outer body from moving proximally relative to the inner body.

8. The catheter insertion device of claim 7, wherein the stop member includes a pair of protrusions extending into the slot, the pair of protrusions forming a passage to an open distal end of the slot; and wherein the rudder of the stabilizer is configured to pass through the passage of the slot upon application of a threshold force against the pair of protrusions to allow the outer body to detach from and/or attach to the inner body.

9. The catheter insertion device of claim 1, wherein a distal end of the outer body comprises a guidewire actuator configured to move the guidewire between the undeployed state and the deployed state.

10. The catheter insertion device of claim 1, further comprising a safety clip configured to provide sharps protection to the tip of the introducer needle upon removal of the catheter from the introducer needle.

11. The catheter insertion device of claim 10, wherein the safety clip comprises a proximal portion comprising a first proximal wall directly connected to a second proximal wall at a clip bend, the first proximal wall defining a first proximal aperture and the second proximal wall defining a second proximal aperture, the second proximal wall being more proximal than the first proximal wall;
a first distal portion comprising a first distal wall, the first distal wall defining a first distal aperture for receiving the needle;
a first clip arm extending from the first distal portion to the first proximal wall and comprising a medial aperture for receiving the needle;
a second distal portion comprising a second distal wall, the second distal wall defining a second distal aperture for receiving the needle;
a second clip arm extending from the second distal portion to the second proximal wall;
the first clip arm and the second clip arm being in contact with the needle, and the first clip arm and the second clip arm are configured to flex between a first configuration and a second configuration to move the safety clip relative to the needle;
wherein, when the first clip arm and the second clip arm are in the first configuration, the safety clip is able to slide along a length of the needle and the first distal wall and the second distal wall are spaced apart;
wherein, when the first clip arm and the second clip arm are in the second configuration, the second proximal aperture is configured to engage a swaged portion of the needle such that the first distal wall and the second distal wall overlap to cover a tip of the needle; and
wherein the first clip arm and the second clip arm do not intersect in either the first configuration or the second configuration.

12. The catheter insertion device of claim 10, wherein the safety clip further comprises a keeper configured to provide frictional resistance against an inner surface of the catheter hub to retain the safety clip inside the catheter hub.

13. The catheter insertion device of claim 10, wherein the catheter hub further comprises a bump configured to prevent the safety clip from being removed from the catheter hub in a first configuration and configured to allow the safety clip to be removed from the catheter hub in a second configuration.

14. A method of operating a device capable of continuous pulsatile blood flash to advance a guidewire fixed to a distal end of an outer body of the device into a vessel of a patient, the method comprising:
moving the outer body of the device to a first position;
inserting a needle attached to a proximal end of an inner body of the device into the vessel of the patient;
moving the outer body from the first position, where a tip of the guidewire is within the needle, to a second position, where the tip of the guidewire is advanced into the vessel of the patient;
wherein when the outer body moves between the first position and the second position, the outer body slides over the inner body and the guidewire moves through an inner portion of the inner body;
wherein the guidewire extends through a passage of a stabilizer and is fixed to a distal end of the inner body; and
wherein the passage is configured to produce a frictional resistance on the guidewire when the outer body moves from the first position to the second position.

15. The method of claim 14, wherein the aperture is further configured to have an axis angularly offset by an angle with respect to a longitudinal axis of the catheter insertion device, wherein the angle is between 3 and 10 degrees.

16. The method of claim 14, wherein the outer body further comprises a stop member at a distal end of an elongated channel configured to abut the projection of the stabilizer to prevent the outer body from moving proximally relative to the inner body.

17. The method of claim 16, wherein the stop member comprises a finger forming a terminus of the elongated channel; and wherein the projection of the stabilizer is configured to elastically deflect the finger upon application of a threshold force on the longitudinal axis of the outer body to allow the outer body to detach from and/or attach to the inner body.

18. The method of claim 14, wherein the outer body further comprises a stop member at a distal end of the elongated slot configured to abut the rudder of the stabilizer to prevent the outer body from moving proximally relative to the inner body.

19. The method of claim 18, wherein the stop member includes a pair of protrusions extending into the slot, the pair of protrusions forming a passage to an open distal end of the slot; and wherein the rudder of the stabilizer is configured to pass through the passage of the slot upon application of a threshold force against the pair of protrusions to allow the outer body to detach from and/or attach to the inner body.

20. The method of claim 19, wherein the catheter insertion device further comprises a safety clip configured to provide sharps protection to the tip of the introducer needle upon removal of the catheter from the introducer needle, wherein when the safety clip reaches a bevel of a tip of the needle, the safety clip activates and allows the first clip arm and the second clip arm to move from the first configuration to the second configuration.
